(19) United States
(12) Patent Application Publication
(10) Pub. No.: US 2025/0352518 A1
(43) Pub. Date: Nov. 20, 2025

(54) A BENZIMIDAZOLE COMPOUND WITH ANTIHELMINTHIC ACTIVITY FOR USE IN REVERSING, ARRESTING OR SLOWING DOWN CELLULAR AGEING IN A VERTEBRATE SUBJECT

(71) Applicant: RAVAN BIO LIMITED, Sale (GB)

(72) Inventors: Ian Noel Hampson, Sale Cheshire (GB); Lynne Hampson, Sale Cheshire (GB); Anthony William Oliver, Congleton (GB)

(73) Assignee: RAVAN BIO LIMITED, Sale (GB)

(21) Appl. No.: 18/872,042

(22) PCT Filed: Jun. 19, 2023
(86) PCT No.: PCT/GB2023/051596

§ 371 Date: Dec. 5, 2024

(30) Foreign Application Priority Data
Jun. 17, 2022 (GB) 2208911.4

(51) Int. Cl. A61K 31/4184; A61K 31/4985; A61P 17/14; A61P 43/00

(52) U.S. Cl. CPC A61K 31/4184; A61K 31/4985; A61P 17/14; A61P 43/00

(57) ABSTRACT

The present inventions concerns the use of compositions comprising a benzimidazole compound with antihelminthic activity for use in reversing, arresting or slowing down cellular ageing in a vertebrate subject. In preferred embodiments the composition may be combined with, or co-administered with, a Phosphodiesterase 5 inhibitor.

Provided herein are methods and compositions for reversing, arresting or slowing down cellular ageing. In particular compositions are provided comprising a benzimidazole compound with antihelminthic activity for use in such methods.

### BACKGROUND

The organs and tissues of animals start to age from the moment of birth and the ageing process is influenced by genetics and lifestyle combined with exposure to biological and chemical agents. The preservation of organ and tissue homeostasis, throughout life, is accomplished by the maintenance of undifferentiated stem-cells throughout the body which have the capacity to either self-renew or terminally differentiate into the different cell types.

When normal stem-cells are extracted from various sites in the body and cultured, they typically go through a limited number of divisions after which they stop dividing and eventually undergo apoptosis which is known as the Hayflick limit. This occurs as a result of cell-division-associated telomere shortening which is regulated by expression of telomerase.

Skin is an example of a self-renewing tissue where the stem-cells reside in the basal layer immediately adjacent to the basement membrane. Indeed, it has recently been shown that it is possible to rejuvenate epidermal stem-cells from aged human skin by maturation-phase transient-reprogramming whereby aged cells were treated for a limited period to induce expression reprogramming factors such as Oct3/4, Sox2, Klf4 and eMyc (e.g. see Gill et al. (2022) Elife April 8:11:e71624. Doi: 10.7554/elife.71624).

The inventors have appreciated that, rather than reprogramming by molecular biology techniques, that it is desirable that small molecules are found that can rejuvenate stem-cells. In fact small molecules are currently being explored as a convenient means of inducing the expression of stem-cell reprogramming factors and progress was reviewed by Lin and Wu in 2015 (Lin and Wu (2015) Stem Cell International Volume 2015 ID794632). However, to date no useful small molecules, which can rejuvenate stem-cells and which have any commercial or clinical value have been established.

Age-related damage to stem-cell populations has a major influence on the health of animals and humans. Such damage promotes the development of multiple disorders such as cancer, cardiovascular, neurodegenerative and autoimmune diseases. The inventors recognized that novel treatments which inhibit cellular ageing have the potential to reduce the development of such ailments simultaneously rather than having to treat each separately. Thus, it is one object of the present invention to provide compounds that will reverse, arrest or slow down cellular ageing in order to reduce the burden of these diseases.

### SUMMARY OF THE INVENTION

The inventors have established that benzimidazole compounds with antihelminthic activity (optionally combined with a Phosphodiesterase 5 inhibitor) are effective for promoting the self-renewal of multiple stem-cell populations throughout a subject's body and therefore such compounds are useful in therapies which will reduce or reverse the ageing process and thereby reduce the incidence or severity of multiple age-related diseases.

According to a first aspect of the invention there is provided a composition comprising a benzimidazole compound with antihelminthic activity for use as a medicament to reverse, arrest or slow down cellular ageing in a vertebrate subject.

According to a second aspect of the invention there is provided a method of reversing, arresting or slowing down cellular ageing in a vertebrate subject in need of such treatment comprising administering a therapeutically effective amount of a composition comprising a benzimidazole compound with antihelminthic activity.

In preferred embodiments of the invention the composition further comprises a Phosphodiesterase 5 inhibitor (PDE 5 inhibitor) and according to a third aspect of the invention there is provided a composition for pharmaceutical, veterinary or nutraceutical use comprising a benzimidazole compound with antihelminthic activity and a PDE 5 inhibitor.

Alternatively, compositions comprising a benzimidazole compound with antihelminthic activity may be co-administered with a composition comprising a PDE 5 inhibitor.

PDE 5 inhibitors have been shown to improve the morphology of ageing skin (see US2013/0030174). However, a use of PDE 5 inhibitors for reversing, arresting or slowing down cellular ageing was not contemplated and most notably, novel combinations of benzimidazole compounds with antihelminthic activity and PDE 5 inhibitors have not been described. The inventors are the first to show synergy between these classes of compounds.

In a preferred embodiment the compounds reduce the epigenetic or biological age of a subject. There are a number of ways this can be measured. In dogs it has been demonstrated that combining DNA based genetic breed determination with analysis of telomere length, can provide a measure of biological age with 95-98% accuracy (Fick et al. (2012) Cell Reports 2 1530-1536) and this is now offered as a service by companies such as EasyDNA using their DNA-My-Dog test. Estimates of epigenetic or biological age in humans can be determined by analysis of DNA methylation markers (Chen et al (2016) Aging 8 (9) p 1844-1859) which is offered as a service by companies such as Elysium with their Index test. The inventors realised that treatment-induced reduction in the biological age of both dogs and humans may subsequently be analysed with these two different methodologies to provide a good basis for assessing the anti-ageing effects of test compounds.

In another preferred embodiment the compounds are used to treat and/or prevent the development of age-related diseases. Examples of age-related diseases include degenerative conditions such as Type II diabetes, skin wrinkles, hair-loss, tooth loss/wear, varicosities, joint-degeneration, muscle loss, neurodegenerative conditions, macular degeneration and decreased immune function.

In another preferred embodiment the compounds are used to treat and/or prevent the development of benign proliferative pathologies such as benign prostatic hyperplasia (BPH), benign proliferative breast diseases, endometriosis, benign proliferative skin growths such as Seborrheic keratosis and lipomas and benign gut pathologies such a polyps.

In another preferred embodiment the compounds are used to prevent the development of malignant proliferative disorders (i.e. cancers).

The inventors believe that their surprising discovery (that compositions according to the invention promote stem cell self-renewal) explains why such compounds are effective for treating and/or preventing age-related degenerative diseases. However, the activity of the compounds for preventing the development of benign or malignant proliferative disorders was considered particularly surprising. With respect to cancer, the inventors appreciate that PDE5 inhibitors and antihelminth benzimidazole compounds have both previously been shown to have activity as anticancer therapeutics against a variety of different types of cancer. However, these compounds have not previously been considered to have any use for cancer prevention. It is important to appreciate that cancer therapy is very different from cancer prevention since the former specifically targets existing cancer cells whereas the latter prevents their formation. The formation of cancer is typically a slow process whereby the evolution of malignant cells usually takes place over several decades during which time, the cells undergo a gradual series of stepwise changes which eventually leads to the emergence of a cancer. These changes are produced by a variety of endogenous and/or exogenous factors although an age-related increase in long-term persistent inflammation, known as “inflammaging”, is a key driver for the development of many cancers. Thus, since the current invention reverses biological ageing, and thereby reduces inflammaging, the inventors believe this prevents cancers from ever forming. Furthermore, since inflammaging also drives benign proliferative disorders in the same way, the current invention should also have activity against these types of pathology.

In another preferred embodiment the compounds are used to promote tissue regeneration following damage produced by either artificial or natural means (e.g. following a spinal injury or burn). The composition also has cosmetic uses for example, the stimulation of tooth repair and teeth whitening.

### BRIEF DESCRIPTION OF THE DRAWINGS

The summary, as well as the following detailed description, is further understood when read in conjunction with the appended drawings. For the purposes of illustrating the disclosed compositions and methods, there are shown in the drawings exemplary embodiments of the compositions and methods; however, the compositions and methods are not limited to the specific embodiments disclosed. In the drawings:

FIG. 1, is a schematic showing the experimental procedure followed in Example 1.

FIG. 2, shows phase contrast images of 60% confluent normal human epithelial keratinocytes (NHEK) cells +/− 10 μM Y27632 (FIG. 2A) and an example of post-confluent NHEK cells (FIG. 2B) as discussed in Example 1.1.

FIG. 3, shows phase contrast images of re-cultured post-confluent NHEK cells that were treated as specified and as discussed in Example 1.2.

FIG. 4, shows phase contrast images of re-cultured post-confluent NHEK cells that were treated as specified and as discussed in Example 1.2.

FIG. 5, shows phase contrast images of re-cultured post-confluent NHEK cells that were treated as specified and as discussed in Example 1.3.

FIG. 6, shows phase contrast images of passage 14 NHEK cells that were treated as specified and as discussed in Example 1.4 (FIG. 6a); and a bar chart showing the viability of the cells (FIG. 6b).

FIG. 7, shows a short term colorimetre growth analysis of NHEK cells treated as specified and as discussed in Example 1.5

FIG. 8, shows the population expansion together with rate of telomere loss after repeated passage of NHEK cells treated as specified and as discussed in Example 1.6 (FIG. 8a and FIG. 8b)

FIG. 9, shows IL-6 secretion by NHEK cells treated as specified and as discussed in Example 1.7 with sub-confluent (FIG. 9a) and post-confluent (FIG. 9b) cells.

FIG. 10, shows relative telomere length in DNA extracted from cheek epithelial cells from two human case studies immediately after, and 8 months post-treatment, as discussed in Example 3.3.

FIG. 11, shows relative telomere length in DNA extracted from buccal epithelial cells isolated from 60 rodents treated as specified for 4 months as discussed in Example 4.

### DETAILED DESCRIPTION

The disclosed compositions and methods may be understood more readily by reference to the following detailed description taken in connection with the accompanying figures, which form a part of this disclosure. It is to be understood that the disclosed compositions and methods are not limited to the specific compositions and methods described and/or shown herein, and that the terminology used herein is for the purpose of describing particular embodiments by way of example only and is not intended to be limiting of the claimed compositions and methods.

Reference to a particular numerical value includes at least that particular value, unless the context clearly dictates otherwise. When a range of values is expressed, another embodiment includes from the one particular value and/or to the other particular value. Further reference to values stated in ranges, include each and every value within that range. All ranges are inclusive and combinable.

It is to be appreciated that certain features of the disclosed compositions and methods which are, for clarity, described herein in the context of separate embodiments, may also be provided in combination in a single embodiment. Conversely, various features of the disclosed compositions and methods that are, for brevity, described in the context of a single embodiment, may also be provided separately or in any sub-combination.

The following abbreviations are used herein: Active Pharmaceutical Ingredient (API); phosphodiesterase type 5 (PDE5); normal human epithelial keratinocytes (NHEK); Keratinocyte Serum Free Medium (KSFM); Fibroblast Growth Factor (FGF).

As used herein, the singular forms “a.” “an,” and “the” include the plural.

When values are expressed as approximations, by use of the antecedent “about,” it will be understood that the particular value forms another embodiment. Furthermore, the term “about” when used in reference to numerical ranges, cut-offs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of =0.5% or less, or variations of ±0.1% or less from the specified value.

As used herein, by the terms “reverse” or “reversing”, when compared to untreated controls, we mean, at least one selected from: resumed growth of post-mitotic cell-cell contact growth-arrested cells or post-confluent cells: an increase of telomere length; reduced age-associated DNA methylation markers; and/or regression of age associated pathologies.

As used herein, by the terms “arrest” or “arresting”, when compared to untreated controls, we mean, at least one selected from: continued growth of normal human stem cells: stopping a decrease of telomere length over time: no increase in age-associated DNA methylation markers; and/or stabilisation of age-associated pathologies.

As used herein, by the terms “slow down” or “slowing down” when compared to untreated controls, we mean, at least one selected from: a slower reduction in continuous growth of normal human stem cells; a slower decrease of telomere length: a slower increase in age-associated DNA methylation markers; and/or slower development of age-associated pathologies.

As used herein, “treating” and like terms refer to reducing the severity and/or frequency of symptoms, eliminating symptoms and/or the underlying cause of said symptoms, reducing the frequency or likelihood of symptoms and/or their underlying cause, delaying, preventing and/or slowing the progression cancers or benign proliferative disorders, and improving or remediating damage caused, directly or indirectly, by the cancers or disorders.

As used herein, the phrase “therapeutically effective dose” refers to an amount of a composition comprising a composition comprising a benzimidazole compound with antihelminthic activity as described herein, effective to achieve a particular biological or therapeutic result such as, but not limited to, biological or therapeutic results disclosed, described, or exemplified herein. The therapeutically effective dose may vary according to factors such as the disease state, age, sex, and weight of the individual, and the ability of the composition to cause a desired response in a subject. Such results include, but are not limited to, reversing, arresting or slowing down cellular ageing as determined by any means suitable in the art.

As used herein, “vertebrate subject” includes any animal of veterinary interest (e.g. sheep, cattle, horses, fish, dogs, cats, rabbits and most reptiles) and also humans. Preferably the subject is a mammal. In one embodiment of the invention the subject may be a domestic animal (e.g. a dog or cat) or animal used in leisure or sporting pursuits (e.g. a horse). In another embodiment of the invention the subject may be a farm animal. In a preferred embodiment of the invention the subject is a human being.

As used herein “a benzimidazole compound with antihelminthic activity” means a compound containing the structure of formula I (see below) that has activity as an antiparasitic drug for expelling or killing parasitic worms (helminths) and other internal parasites from a subject. Antihelminthics may be used to treat humans who are infected by helminths and may also be used to treat other vertebrate subjects.

As used herein, reference to the Active Pharmaceutical Ingredient (API) means a benzimidazole compound with antihelminthic activity and/or a PDE 5 inhibitor depending on the context.

As used herein, “post-confluent cells” refers to normal cells grown past confluence which undergo irreversible growth-arrest and senescence due to contact inhibition. The cells can subsequently be passaged and cultured in a suitable vessel and used as models of aged cells.

A “pharmaceutically acceptable vehicle” may be any physiological vehicle known to those of ordinary skill in the art useful in formulating pharmaceutical compositions.

The inventors recognized that cell lines from vertebrates may be used as a model for screening compounds to assess whether or not a compound is capable of reversing, arresting or slowing down cellular ageing. They developed a model which involved allowing an adherent cell line to grow in a flask (or the like) to confluence. At this stage, cells typically stop dividing, become post-confluent and ultimately undergo apoptosis. Such cells have effectively reached their Hayflick limit. The inventors were inspired to disperse and continue to culture such post-confluent cells. A skilled person would expect such cultures to fail because the Hayflick limit has been reached. However, the inventors hypothesized that compounds capable of reversing, arresting or slowing down cellular ageing may be expected to improve survival of such cultures.

The inventors decided to use epidermal stem-cells as a proxy for other metazoan stem-cell populations in their model for identifying compounds with general anti-ageing properties (see Example 1). Skin is the largest organ in the human body and as a subject ages it thins, loses elasticity, becomes fragile and less able to heal. It is composed of stratified layers of cells which, starting from the outermost layer, are known as the stratum corneum, stratum lucidum, stratum granulosum, stratum spinosum and the stratum basale, which is where the basal epidermal stem-cells reside.

Significantly, age-related deterioration of skin is considered to be due to a reduction in both the number and quality of basal stem cells. These cells either undergo self-renewal by symmetrical division forming two identical stem-cells, or asymmetrical division where the product is one stem-cell and one differentiating supra-basal cell. Clearly the former is more efficient at maintaining the stem-cell population whereas the latter becomes more prevalent in ageing skin. The inventors grew epidermal cells to confluence and verified that the cells had terminally differentiated with evidence of no growth or minimal stem cells in the culture. They then used such post-confluent cells as models of aged skin and investigated whether or not compounds could reverse, arrest or slow down aging of such cells.

It has recently been shown that increased adhesion of basal stem-cells to the basement membrane is induced by expression of higher levels of collagen 17A1 and this is associated with increased numbers of symmetrical divisions. Most notably, this effect was also shown to be induced by treatment with the Rho Kinase (ROCK) inhibitor Y27632 (Lui et al (2019) Nature 568 p 344-350). Y27632, which is not suitable to be licensed as a drug, has been shown to induce reversible immortalization of neonatal human epidermal foreskin keratinocytes and to also prolong the lifespan of cultures of adult human keratinocytes by extending the Hayflick limit. Indeed, the use of Y27632 and other ROCK inhibitors, in enabling the growth, expansion and differentiation of a wide variety of different stem cell types, including human embryonic stem cells, is now well established and a list of such factors, including Y27632, can be found in Table 1 of Lin & Wu (supra) and (Amand et al (2016) J Biol Methods 23; 3 (2): e41. Doi: 10.14440/jbm.2016.110.eCollections 2016)

The inventors decided to use Y27632 as a positive control in their model. They compared the effect of test compounds on the growth of cultured human N-Tert keratinocytes with untreated cells (a negative control for which no or minimal growth of post-confluent cells was expected) and with Y27632 treated cells (for which some improvement in the growth of post confluent cells was expected). The work conducted is described in detail in Example 1.

The present invention is based on the inventors surprising observation that the benzimidazole antihelminth compound Fenbendazole, and also the PDE5 inhibitor Tadalafil, promoted the growth of the post-confluent cultured human N-Tert keratinocytes. Thus, in spite of both of these two compounds having very different molecular targets to Y27632, the inventors realized that benzimidazole compounds with antihelminthic activity and PDE 5 inhibitors were capable of delaying the Hayflick limit, promoting self-renewal and enhancing cell division and were therefore of veterinary/clinical use for treating subjects where reversing, arresting or slowing down cellular ageing would be beneficial.

The work of Gill et al. (supra) on induced rejuvenation of epidermal stem-cells, supports wider applications of the findings contained herein and the inventors developed their model (see Example 1) as a proxy for more general efficacy on stem-cell populations present in other organs and tissues in vivo (in addition to the skin). An April 2022 news article (https://www.bbe.co.uk/news/science-environment-60991675) has reported that scientists at the Babraham Institute in Cambridge (UK) have managed to rejuvenate skin cells from a 53 year old woman using undisclosed induced Pluripotent stem (iPS) cell techniques (based on the 1990s techniques used in the creation of “Dolly the sheep”) to induce genetic changes in adult cells. The Cambridge group suggested their work could lead to the treatment of a range of conditions where reversing, arresting or slowing down cellular ageing could be beneficial. However, they have not disclosed what agents may be used. Furthermore, not only do they stress that their work is early stage, but the report also suggests that is not an obvious conclusion that their work could lead to an “anti-aging pill”. Professor Robin Lovell-Badge, of the Crick Institute in London, stated in the same report that he believes the scientific hurdles between this work and even the simplest clinical applications are considerable. Nor does he think it will be a trivial process to translate the rejuvenation process to other types of tissue or indeed produce an anti-ageing pill. Thus, it could not be predicted that any compositions they may contemplate would possess general anti-aging properties and the report certainly does not disclose or suggest the utility of benzimidazole compounds with antihelminthic activity and/or a PDE 5 inhibitor as contemplated herein.

Having established the effects of compounds according to the invention in vitro, the inventors next investigated the effect of the compounds in canines by measuring the biological age of animals using a commercial test following treatment (see Example 2). The test was based on measuring telomere length of canine cell samples as reported by Fick et al. (Cell reports (2012) Vol 2 (6) p 1530-1536). When Fenbendazole was administered over a 4-5 year period, there was a surprising 80% reduction in biological age when compared to chronological age. Furthermore, age-related pathologies were also seen to be reduced in these animals; there was increased growth of hair; and, most surprisingly, regrowth and regeneration of teeth. Notably, the observations on enhanced growth of teeth would not be apparent in rodents since, unlike dogs, they have open-rooted dentition which means their teeth grow continuously throughout their entire lifespan.

Further case studies assessed epigenetic age before and/or after a course of treatment with compounds according to the invention combined with assessment of age-related pathologies such as osteoarthritis, cardiac artery stenosis, benign prostatic hyperplasia, etc.

The Examples illustrate that compounds used according to the invention may be used to reverse, arrest or slow down cellular ageing. The compounds are particularly useful for:
(a) reducing the epigenetic or biological age of a subject;
(b) treating and/or preventing age-related degenerative diseases such as Type II diabetes, skin wrinkles, hair-loss, tooth loss/wear, joint-degeneration, muscle-loss, varicosities, cardiovascular diseases, neurodegenerative conditions, macular degeneration, decreased immune function and potentially alleviating the symptoms of menopause;
(c) treating benign proliferative disorders and preventing cancer;
(d) promoting tissue regeneration of damage produced by either artificial (e.g. following spinal injury, burn, radiotherapy, chemotherapy, infections) or natural means (normal wear and tear).

It is to be appreciated that the preceding list of age-related conditions is not exhaustive and it is anticipated that the compounds used, according to the invention, may have activity against numerous other age-related pathologies that are not listed.

The data provided in Example 1 demonstrates how APIs, according to the invention, are superior to Y27632 in promoting continued growth of cell-cell contact growth arrested cells and that Fenbendazole is the most potent in this regard. The inventors realised that the ability to rejuvenate epidermal stem cells indicates such APIs will have a more general ability to rejuvenate stem cells from other tissue types which leads to more profound effects in vivo. Historically, the inventors conceived the idea that Fenbendazole may possess anti-ageing properties >4 years ago and, at the time, embarked on a long-term treatment program in dogs to assess this by examining physical markers of ageing (hair greying, mobility, teeth discolouration etc) as discussed in Example 2. However, during this period, biological age-testing of dogs became commercially available and this was carried out on the two indicated canine subjects post-treatment with Fenbendazole. The first 10 yr old subject was treated for a 4 year period until aged 14 yrs and showed an 80% reduction in biological age when compared to chronological age which clearly supports the uses described in (a), (b) (c) & (d) above. The other subject commenced treatment at the same time but was younger than the first (4 yrs old). This dog was given a higher-dose, more intensive treatment for 19 months which was then discontinued for 2 yrs 5 months. Biological age-assessment of this dog was carried out at a chronological age of 8 yrs and showed an age of 1 yr old which corresponds to ˜90% reduction in biological age which also clearly supports the uses described in (a), (b), (c) & (d).

### APIs Used According to the Invention

Benzimidazole compounds with antihelminthic activity used according to the invention comprise a benzimidazole structure of formula I with substitutions at positions 2, 5 and/or 6 of the structure of formula I.

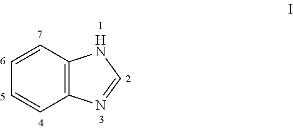

Benzimidazoles compounds used according to the invention are preferably selected from the group comprising: Fenbendazole, Mebendazole, Flubendazole, Parbendazole, Oxfendazole, Oxibendazole, Albendazole, Ricobendazole, Albendazole sulfoxide, Thiabendazole, Thiophanate, Febantel, Netobimin, and Triclabendazole.

It is more preferred that the benzimidazole used according to the invention is selected from the group comprising Triclabendazole, Mebendazole and Fenbendazole and functional analogues thereof. These inhibitors have the structures set out in formula II-V.

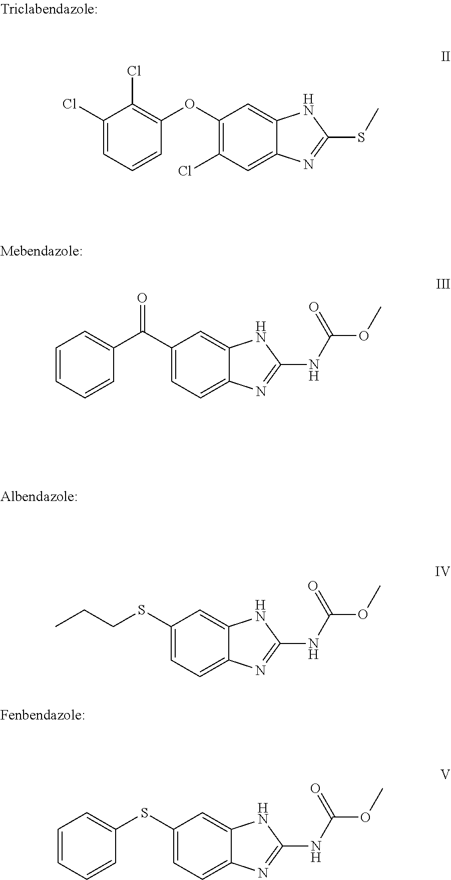

It is most preferred that the benzimidazole compound used according to the invention is fenbendazole (Formula V) or a structural equivalent thereof. The chemical name of fenbendazole is methyl 5-(phenylthio)-2-benzimidazole carbamate (CAS Registry Number is 43210-67-9).

A structural equivalent of fenbendazole may have a drugbank similarity threshold (Tanimoto Score) of 0.6; more preferable of 0.7 and most preferably of 0.8.

The biological effects of the benzimidazole compounds are complex which makes identification of their mode-of-action for reversing, arresting or slowing down cellular ageing difficult to define. It is notable benzimidazole compounds have been proposed for the treatment of cancer and there have been numerous mechanistic studies carried out on this which have identified a broad range of pleiotropic effects (Son et al. (2020) Immune Netw 20 (4): e29). However, it is counterintuitive that compounds with anticancer properties (where cell proliferation is undesirable) would also have activity against the ageing process (where cell proliferation is desirable) as disclosed herein. This view is reinforced by the observation, in cancer cells, that benzimidazole compounds have been shown to reduce expression of stem cell markers and increase expression of keratinocyte differentiation factors (p6/20 of Son et al. supra). Accordingly, an anti-ageing activity is surprising, and an activity for cancer prevention is particularly surprising, in view of Son et al because the obverse would be expected. Although the inventors do not wish to be bound by any hypothesis, they have noted the reported ability of benzimidazole compounds to reduce expression of pro-inflammatory SASP factors such as Cox-2, TNF-α and IL-6 and believe this anti-inflammatory effect may contribute to the anti-senescent/anti-ageing activity disclosed herein (See Example 1).

In preferred embodiments compositions according to the invention may comprise a benzimidazole compound combined with a phosphodiesterase type 5 inhibitor (PDE5 inhibitor). In other preferred embodiments the composition according to the first aspect of the invention may be co-administered with a composition comprising a PDE5 inhibitor.

PDE5 inhibitors are vasodilators which work by preventing the degradative action of cGMP-specific phosphodiesterase type 5 (PDE5) on cyclic GMP in smooth muscle cells lining the blood vessels supplying various tissues. Their medical uses include the treatment of erectile dysfunction, pulmonary hypertension and benign prostatic hyperplasia.

PDE5 Inhibitors that may be used according to the invention are preferably selected from the group comprising vardenafil, sildenafil, avanafil, tadalafil and functional equivalents thereof. These inhibitors have the structures set out in formula VI-IX.

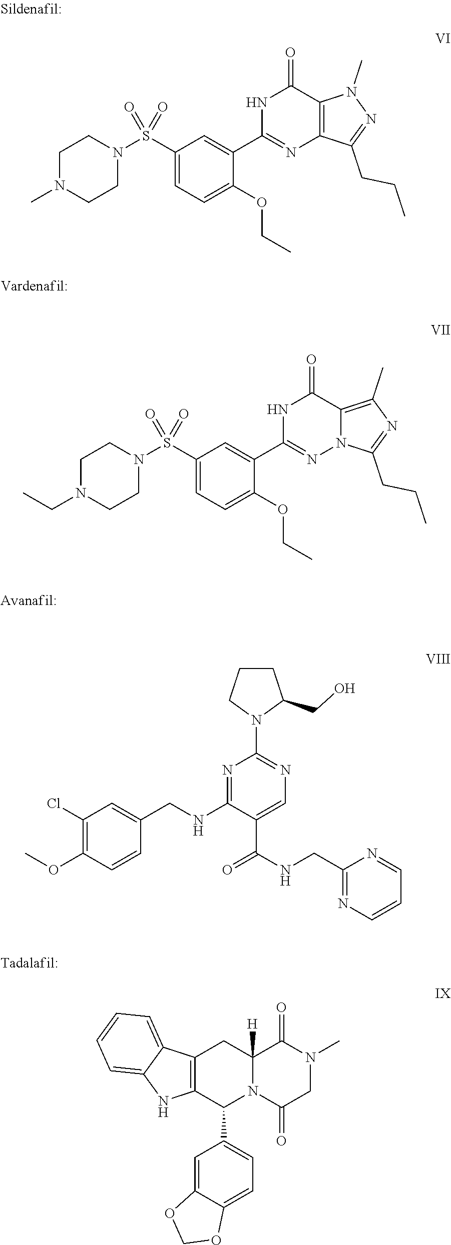

It is most preferred that the PDE5 inhibitor used according to the invention is tadalafil (Formula IX) or a structural equivalent thereof. The chemical name of tadalafil is pyrazino[1′,2′: 1,6]pyrido[3,4-b]indole-1,4-dione, 6-(1,3-benzodioxol-5-yl)-2,3,6,7,12,12a-hexahydro-2-methyl-, (6R,12aR).

A structural equivalent of tadalafil may have a drugbank similarity threshold (Tanimoto Score) of 0.6: more preferrable of 0.7 and most preferably of 0.8.

The inventors have noted that the ageing process is known to be related to oxidative damage and believe PDE5 inhibitors may be useful according to the invention because they have been shown to improve reactive oxygen (ROS) metabolism in skeletal muscle cells and may also enhance the capacity of enzymatic antioxidant systems (Duranti et al. (2017) Cell Stress and Chaperones 22:389-396). Furthermore, PDE5 inhibitors have been shown to reduce ROS-induced DNA damage in human dermal fibroblasts isolated from systemic sclerosis patients which counteracts the negative effects of ROS on cell viability and proliferation by promoting the activity of specific enzymes involved in the maintenance of redox homeostasis. It is also significant that sildenafil significantly reduced expression and release of IL-6 from systemic sclerosis dermal fibroblasts and IL-6 is a key marker of inflammatory changes and the senescence associated secretory phenotype (SASP) (Luigi et al (2020) Int J Mol Sci 21, 3161; doi:10 2290/ijms21093161). Indeed, upregulated IL-6 is known to be associated with chronic age-associated inflammation and, while the inventors do not wish to be bound by any hypothesis, anti-inflammatory effect may also contribute to the anti-senescent activity of PDE5 inhibitors.

A most preferred composition according to the third aspect of the invention may comprise Fenbendazole and Tadalafil.

In other preferred embodiments of the invention a composition according to the first aspect of the invention comprising Fenbendazole may be co-administered with a composition comprising Tadalafil.

Compositions according to the invention may be used in combination with other licensed drugs that are known to inhibit or reverse biological ageing in animals and human. For instance, the compositions may be used with Rapamycin (and its Rapalogs), Dasatinib, Metformin or Novitoclax.

Compositions according to the invention may also be used in combination with nutraceuticals or nutritional supplements that are known to inhibit or reverse biological ageing in animals and human. For instance, the compositions may be used with Fisetin, Resveratrol, Quercetin, Coenzyme Q10, Berberine, Curcumin, N-Acetyl Cysteine, Alpha-Lipoic-Acid, Alpha-Ketoglutarate, Apigenin, Vitamin C, Vitamin E, Vitamin D3, Retinol or Retinoic acid, Nicotinamide Mononucleotide, Omega-3-fish oil, or plant based polyphenols.

### Pharmaceutical and Nutraceutical Formulations

The compositions used according to invention may comprise the API or APIs without any additional components (e.g. a powder of the API which is used by diluting in a liquid or used to fill a capsule). However, in preferred embodiments the APIs are formulated with other agents, as discussed below, to improve their commercial properties (e.g. to improve delivery, shelf-life, taste and the like).

### Compositions for Oral Administration

The compositions of the invention may be formulated as a pharmaceutical or nutraceutical composition for oral administration. As such, they can be formulated as gels, solutions, suspensions, syrups, tablets, capsules, lozenges and snack bars or beverages by way of example. Such formulations can be prepared in accordance with methods well known to the art. For example, the API or APIs may be formulated in a syrup or other solution for administration orally, for example as a health drink. One or more excipients selected from sugars, vitamins, flavouring agents, colouring agents, preservatives and thickeners may be included in such syrups or solutions. Tonicity adjusting agents such as sodium chloride, or sugars, can be added to provide a solution of a particular osmotic strength. One or more pH-adjusting agents, such as buffering agents can also be used to adjust the pH to a particular value, and preferably maintain it at that value. Examples of buffering agents include sodium citrate/citric acid buffers and phosphate buffers.

In preferred embodiments the API or APIs are formulated as a tablet for oral consumption. For tablet formation, the API or APIs may be typically mixed with a diluent such as a sugar, e.g. sucrose and lactose, and sugar alcohols such as xylitol, sorbitol and mannitol; or modified cellulose or cellulose derivative such as powdered cellulose or microcrystalline cellulose or carboxymethyl cellulose. The tablets will also typically contain one or more excipients selected from granulating agents, binders, lubricants and disintegrating agents. Examples of disintegrants include starch and starch derivatives, and other swellable polymers, for example crosslinked polymeric disintegrants such as cross-linked carboxymethylcellulose, crosslinked polyvinylpyrrolidone and starch glycolates. Examples of lubricants include stearates such as magnesium stearate and stearic acid. Examples of binders and granulating agents include polyvinylpyrrolidone. Where the diluent is not naturally very sweet, a sweetener can be added, for example ammonium glycyrrhizinate or an artificial sweetener such as aspartame, or sodium saccharinate.

The API or APIs can also be formulated as powders, granules, gels or semisolids for incorporation into capsules. When used in the form of powders, the API can be formulated together with any one or more of the excipients defined above in relation to tablets, or can be presented in an undiluted form. For presentation in the form of a gel or semisolid, the API or APIs can be dissolved or suspended in a viscous liquid or semisolid vehicle such as a polyethylene glycol, or a liquid carrier such as a glycol. e.g. propylene glycol, or glycerol or a vegetable or fish oil, for example an oil selected from olive oil, sunflower oil, safflower oil, evening primrose oil, soya oil, cod liver oil, herring oil, etc. These can then be filled into capsules of either the hard gelatine or soft gelatine type or made from hard or soft gelatine equivalents, soft gelatine or gelatine-equivalent capsules being preferred for viscous liquid or semisolid fillings. In one preferred embodiment, a composition according to the invention is provided in powder form optionally together with a preferred solid (e.g. powdered) excipient for incorporation into capsules, for example a hard gelatine capsule.

### Preferred Formulations for Oral Administration

It is preferred that benzimidazole compounds with antihelminthic activity used according to the invention are formulated for oral consumption as a powder (for mixing with food or making into a drink), granules, aqueous suspensions, pastes, tablets or capsules.

In one embodiment of the invention the composition according to the invention comprises 200-500 mg Fenbendazole in a tablet formulated with anhydrous colloidal silica, corn starch, sodium carboxymethyl starch (type A), hydroxyethylcellulose, lactose monohydrate and Magnesium Stearate.

In another, embodiment of the invention a most preferred formulation for human use is a capsule essentially containing pure Fenbendazole. Such capsules may comprise: 100-500 mgs fenbendazole; preferably 150-300 mgs Fenbendazole; and more preferably 200-250 mgs fenbendazole. In a preferred embodiment the capsule comprises about 222 mgs fenbendazole.

In a further embodiment of the invention Fenbenzadole may be taken as solid granules which may be mixed with food and then consumed. By way of example, granulated formulations are marketed as Panacur® for use as an antihelminthic for treating animals. Panacur® Granules containing 22.2% (w/w) Fenbendazole in addition to the excipients lactose monohydrate, Povidone 2500 and maize starch. These granules are used as a dewormer for cats or dogs (by mixing Panacur® granules with their food) and may be repurposed and/or adapted for use according to the present invention.

In a further embodiment of the invention Fenbendazole may be formulated as a suspension (to be drunk or mixed with food). Fenbendazole is also available as Panacur® Equine Guard aqueous 10% w/v oral suspension combined with the excipients Sodium Methyl Parahydroxybenzoate, Sodium Propyl Parahydroxybenzoate, Benzyl alcohol, Silica colloidal anhydrous, Carmellose sodium, Povidone K25, Sodium Citrate Dihydrate, Citric Acid Monohydrate and Water Purified. It is used for treating horses and may be repurposed and/or adapted for use according to the present invention.

In a further embodiment of the invention Fenbendazole may be formulated as a paste. Panacur® is also available as an 18.75% (w/w) Fendendazole paste combined with the excipients Methyl Parahydroxybenzoate, Propyl Parahydroxybenzoate, Propylene Glycol, Apple and Cinnamon Flavour, Carbomer 980, Glycerol (85%), Sorbitol (70%, crystallising), Sodium Hydroxide, Water Purified. It is used for administering orally (by syringe) to rabbits and the like and may be repurposed and/or adapted for use according to the present invention.

When PDE 5 inhibitors are used as an API, the inhibitors may also be formulated as a powder (for mixing with food or making into a drink), granules, aqueous suspensions, pastes, tablets or capsules. In a preferred embodiment PDE 5 inhibitors are formulated as tablets for oral consumption. By way of example, tablets for oral delivery comprising 2.5, 5, 10, or 20 mg of Tadalafil are known to the art for treating erectile dysfunction in humans. An example of such tablets comprises Tadalafil and the following inactive ingredients: croscarmellose sodium, hydroxypropyl cellulose, hypromellose, iron oxide, lactose monohydrate, magnesium stearate, microcrystalline cellulose, sodium lauryl sulfate, talc, titanium dioxide, and triacetin. It will be appreciated that such formulations may be used or adapted for treating animal or human subjects according to the invention and co-administered with a benzimidazole with antihelmintic activity.

The abovementioned benzimidazole compound formulations, and particularly the Panacur® formulations, may be adapted to co-formulate both benzimidazoles, such as Fenebendazole, and PDE5 inhibitors, such as Tadalafil in the same composition. This could be at ratios ranging from 1:1 to 1000:1 w/w Fenbendazole/Tadalafil.

### Dosing and Dose Units for Oral Use

The amount of a composition used to treat subjects according to the invention will depend upon the species being treated; their size, age and sex: the specific condition being treated or prevented; and the severity of the condition being treated. The required amount can be presented in the form of a unit dosage form containing a defined amount of the API or APIs.

Benzimidazole compounds with antihelminthic activity may be administered as an oral dose of between 0.1 and 50 mg/Kg/day. It is preferred that a subject receives between 0.5 and 20 mg/Kg/day and more preferred subject receives between 1 and 15 mg/Kg/day. By way of example a canine or human subject may benefit from receiving between about 2 and 15 mg/Kg/day.

As single agents, PDE5 inhibitors may be administered as an oral dose of between 1 and 600 ng/Kg/day). It is preferred that a subject receives between 5 and 300 ng/Kg/day and more preferred subject receives between 10 and 150 ng/kg/day. By way of example a human subject may benefit from receiving between about 30 and 100 ng/Kg/day with an equivalent dose used for canines.

Treatment with compositions of Fenbendazole, according to the first aspect of the invention, may be given as a single agent or be used in conjunction with PDE5 inhibitors also given as either single agents, or co-formulated at ratios of between 1:1 and 1000:1 w/w. More preferably this will be at ratio of between 10:1 and 100:1 and most preferred between 20:1 and 70:1.

The compositions according to the invention can be presented in the form of unit dosage forms containing a defined amount of the API or APIs. Such unit dosage forms can be selected so as to achieve a desired level of biological activity and/or deliver the daily amounts discussed above. The amount required in a dose unit will depend up the species being treated: their size, age and sex; and the condition being treated.

By way of example, a unit dosage form of a benzimidazole compound with antihelminthic activity for oral consumption by humans can contain an amount of up to 1000 mg (dry weight) of the API, more typically up to 300 mg, for example between 200 mg and 400 mg.

Particular amounts of the benzimidazole compound may be included in a unit dosage form and may be selected from 50 mg, 75 mg, 100 mg, 150 mg, 200 mg, 250 mg, 300 mg, 350 mg, 400 mg and 450 mg.

A unit dosage form of a PDE5 inhibitor for oral consumption by humans can contain an amount of up to 100 mg (dry weight) of the API, more typically up to 50 mg, for example 0.5 mg to 50 mg. Particular amounts of the PDE5 inhibitor may be included in a unit dosage form may be selected from 1 mg, 1.5 mg, 2.0 mg, 2.5 mg, 5 mg, 7.5 mg, 10 mg, 15 mg, 20 mg, 25 mg, 30 mg, 35 mg, 40 mg and 45 mg

In one embodiment of the invention, humans may be treated with separate compositions or co-formulated compositions providing about a 5 mg/day dose of Tadalafil and about a 200 mg/day of Fenbendazole.

### Compositions for Topical Administration

Compositions according to the invention may also be formulated as a medicament or functional cosmetic that is suitable for topical application and may in particular be formulated for administration to the skin.

Suitable formulations include, but are not limited to, a gel, cream, paste, ointment or lotion. In some aspects, the composition can be formulated as a gel. In some aspects, the composition can be formulated as a cream. In some aspects, the composition can be formulated as a paste. In some aspects, the composition can be formulated as an ointment. In some aspects, the composition can be formulated as a lotion.

In preferred embodiments, the composition is formulated such that it is suitable for topical delivery of the APIs (e.g. as an ointment, gel, paste lotion or cream) for reversing, arresting or slowing down cellular ageing.

When used to reverse, arrest or slow down dermal ageing, the compositions can be formulated as gels, lotions, paste, creams or ointments that may be applied directly to the skin by techniques known to the art.

Preferred compositions for use according to the invention are formulated for topical application to the skin and may comprise fenbendazole.

In one embodiment, the pharmaceutically acceptable vehicle can be a liquid and the composition can be a solution. In another embodiment, the vehicle can be a gel and the composition can be a gel for applying to the skin. In a further embodiment, the vehicle can be an emulsion (or other pharmaceutically acceptable base) and the composition can be a cream. In a further embodiment, the vehicle can be smooth and oily and the composition can be an ointment for application to the skin.

Liquid vehicles may be used in preparing gels, lotions, creams, solutions, suspensions and emulsions. The active ingredient can be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilizers, emulsifiers, buffers, preservatives, suspending agents, thickening agents, colours, viscosity regulators, stabilizers or osmo-regulators. Suitable examples of liquid vehicles include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). The vehicle can also be an oily ester such as ethyl oleate and isopropyl myristate. The liquid vehicle for pressurized compositions can be halogenated hydrocarbon or other pharmaceutically acceptable propellant.

### Uses of Compositions According to the Invention

Age-related damage to, or age-related diminution of, stem-cell populations has a major influence on human and animal health and promotes the development of multiple disorders as described at (a), (b), (c) & (d) (above).

The inventors believe that novel treatments which inhibit cellular ageing have the potential to reduce the development of many ailments simultaneously rather than having to treat each separately. Work carried out by the inventors has established that benzimidazole compounds with antihelminthic activity as single agents, and in preferred embodiment combined with PDE 5 inhibitors, reverse, arrest or slow down cellular ageing in order to reduce the burden of these diseases.

In a preferred embodiment the composition or compositions reverse, arrest or slow down cellular ageing such that there is a reduction in the epigenetic or biological age of the subject. As discussed previously, the epigenetic or biological age of a subject is preferably determined by measuring telomere length or analyzing the extent of specific DNA methylation markers.

In another preferred embodiment the composition or compositions reverse, arrest or slow down cellular ageing results such that an age-related disease or condition, as defined in (a), (b), (c) & (d), are treated or prevented.

In another preferred embodiment the composition or compositions reverse, arrest or slow down the consequences of cellular ageing such that regeneration of damaged tissue, following injury, is promoted (e.g. following spinal injury, a burn or tooth damage).

### Human Use

Due to scientific and technological advances, humans are living longer than ever before. Unfortunately, this increased life expectancy has been accompanied by an increase in many age-related disorders including cardiovascular, neurological and autoimmune diseases. Indeed, the current COVID-19 pandemic has highlighted age-related increased susceptibility to viral infections, which occurs in older people. It is very significant that recent scientific studies have clearly shown that a major aspect of the pathology, underlying all of these conditions, is related to a build-up of pro-inflammatory ‘worn-out’ or senescent cells due to the ageing process. Historically, cellular senescence is defined as an irreversible state where cells can no longer multiply although recent discoveries in this field have produced new insights into the more sinister properties of such worn-out cells. Instead of being removed, these damaged cells accumulate and produce a plethora of pro-inflammatory factors which can damage both stem-cells and other differentiated cell types causing them to become senescent. The inventors believe that this, in turn, promotes the development of the aforementioned different age-related diseases.

Compositions used according to the invention are beneficial when administered to people of all ages.

In one embodiment compositions used according to the invention are beneficial in older people. Typically the compositions will be of benefit to people over the age of 50.

Nevertheless, it is important to recognize that compositions used according to the invention will not just be for the benefit of older people. Therefore, in other embodiments compositions used according to the invention are also useful in children and younger adults. Such subjects can be exposed to chemicals and radiation (e.g. as used in cancer therapy). This may artificially accelerates the ageing process and the subject will benefit from being treated as described herein. Furthermore, specific infections, such as HIV, are also known to accelerate ageing in younger people. Accordingly the APIs may benefit HIV +ve subjects.

The inventors have noted that conventional cancer therapies (e.g. radiation and chemotherapy) actually stimulate senescence, depleting stem cells and accelerating the ageing process thereby increasing the production of senescent cells. This, in turn, causes many side effects (e.g. hair loss which can be permanent, infertility etc). Accordingly in a preferred embodiment of the invention the API or APIs may be used to prevent or treat senescence caused by cancer therapies.

In some embodiments of the invention the compounds may be used to prevent the development of cancer. The inventors believe that compositions according to the invention prevent the formation and/or eliminate senescent cells whilst simultaneously promoting the self-renewal of stem cells and thereby providing a revolutionary alternative for preventing cancer and many other age-related disorders. Compositions according to the invention represent a non-toxic treatment which removes these worn-out cells and thereby significantly reduces the chances of cancer developing both in healthy individuals and in surviving cancer patients from all age groups.

In a preferred embodiment that compositions may be used to prevent or treat senescence caused by viruses. For example, the compositions may be used to reduce the development of senescence in the cervical tissues of women that is caused by human papilloma virus (HPV) and thereby reduce the development of benign and cancerous conditions of the cervix. It is also notable that recent work has shown that chemical reduction of the burden of pro-inflammatory senescent cells in older mice, greatly reduces their mortality from coronavirus infection. This provides an explanation for why Covid-19 has a higher death rate in older people and illustrates that compositions according to the invention may be used to treat and/or prevent SARS-COV-2 infection.

### Dose Regimens in Humans

Fenbendazole has been used as an antihelminth treatment in animals for approximately 60 years and the inventors first wondered why owners had not noticed beneficial anti-aging effects in their animals. The inventors realised this is due to the fact that antihelminth treatments are recommended for very short periods. For instance, it is recommended that puppies are typically treated for only 3 consecutive days and this is followed by intervals of between 5, 8, 12 and 16 weeks before any further treatments are given. Furthermore, in adults dogs, deworming can occur on a single occasion and intervals are often longer (for instance it is recommended that PANACUR® is given to adult cats or dogs 2-4 times a year).

In contrast to the short term use of Fenbendazole as an antihelminth, the inventors have found that an anti-ageing effect is optimal if the API is administered as a continuous or discontinuous treatment regimen of 1-7 days/week given for at least 1 month ranging from periods of between 1-12 months in any 12 month period. More preferably, a regimen for using compounds according to the invention involves treating for 2-5 days per week for a continuous period of 1-6 months.

A most preferred regimen involves treatment for 2-5 days per week for a continuous period of 1-3 months; followed by a 1 month treatment interval; and then a repeat of the 1-3 month treatment cycles.

It will be appreciated that there will be differences with respect to how any particular subject may respond to such treatment regimens and some individual tailoring may be required. Such tailoring may be done in the light of repeated epigenetic age-testing combined with assessment of any age-related pathologies.

By way of example a treatment regimen for an adult human male may involve orally administering tablets, capsules, granules, aqueous suspensions or pastes (or the like) containing 200-250 mg Fenbendazole. Administration may be once a day for three to five times a week, with a meal. No fenbendazole is given for the remaining days of the week. This weekly regimen is then repeated for between 1 and 3 months followed by a 1 month treatment interval. Epigenetic age analysis can then be carried out and, based on the outcome, treatment cycles can then either be paused or continued.

Other preferred treat regimens for humans are disclosed in Example 3.

### Veterinary Use

It will be appreciated that veterinary doses and regimens may be adapted from the human regimens discussed above. Examples of regimens used in dogs are provided in Example 2.

PANACUR® (22.2% Fenbendazole) is used for deworming animals and it is recommended that a single dose of proximately 1 g of Fenbedazole is administered per 10 kg bodyweight of a cat or dog. Further treatment is recommended if natural reinfestation occurs and it is advised that treatments should only occur 2-4 times a year.

In contrast to deworming treatments the inventors have found that more frequent treatment will have the beneficial effects described herein.

By way of example, small dogs with a body mass of approximately 9 kg, may be treated with of 0.6 gm/day of 22.5% Fenbendazole granules (PANACUR®). This is equivalent to 15 mg/kg/day of Fenbendazole. Treatment may continue for between 3 and 5 consecutive days/week for 1 month and this cycle repeated every 3^{rd} month providing a total of 4×1 month cycles/annum. The regimen may be continued for a number of years with the overall duration of treatment determined by the use of epigenetic age-testing (DNA-MY-Dog etc). The inventors found that this regimen was useful according to the invention and noted that it represented approximately double the annual dosing recommended for antiworming treatments.

By way of further example, small dogs with a body mass of about 9 Kg can be treated with oral administration of 1 gm/day 22.5% Fenbendazole granules (PANACUR®). This is equivalent to 25 mg/kg/day of Fenbendazole. Treatment may continue for between 3 and 5 consecutive days per week continuously for between 3-6 months after which the dose is reduced to 0.6 gm/day of PANACUR® (15 mg/Kgm of Fenbendazole) and treatment alternated between 1 month with and 1 month without for a period determined by epigenetic age-testing. Once the desired reduction in biological age is achieved, treatment can be discontinued for periods of 1-3 years followed by regular biological age-testing as a means of estimating efficacy and when further treatment cycles may be required. The inventors found that this regimen was useful according to the invention and noted that it represented over four times the annual dosing recommended for antiworming treatments.

### Example 1

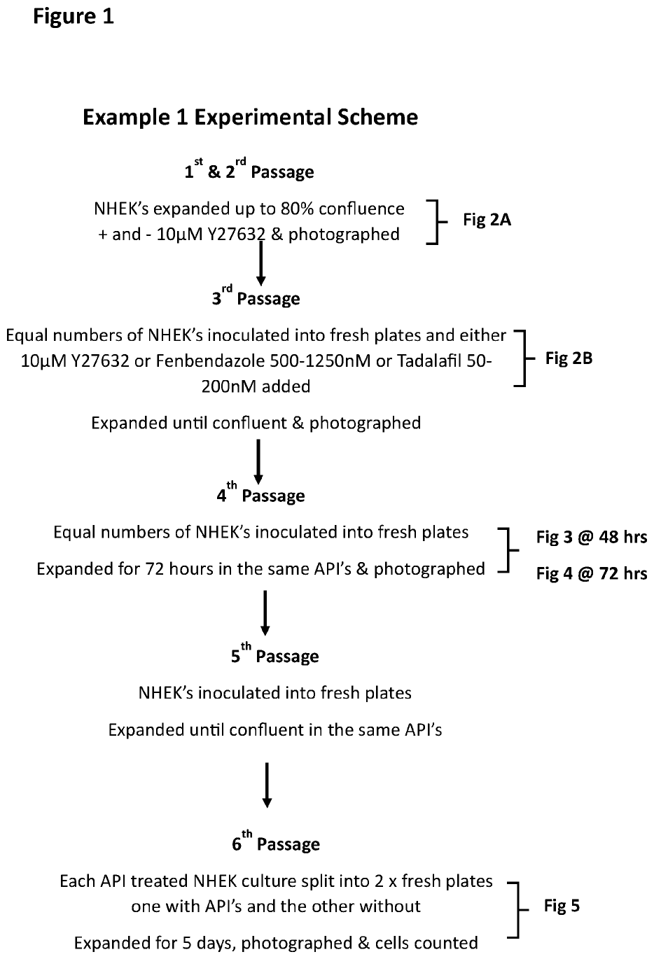

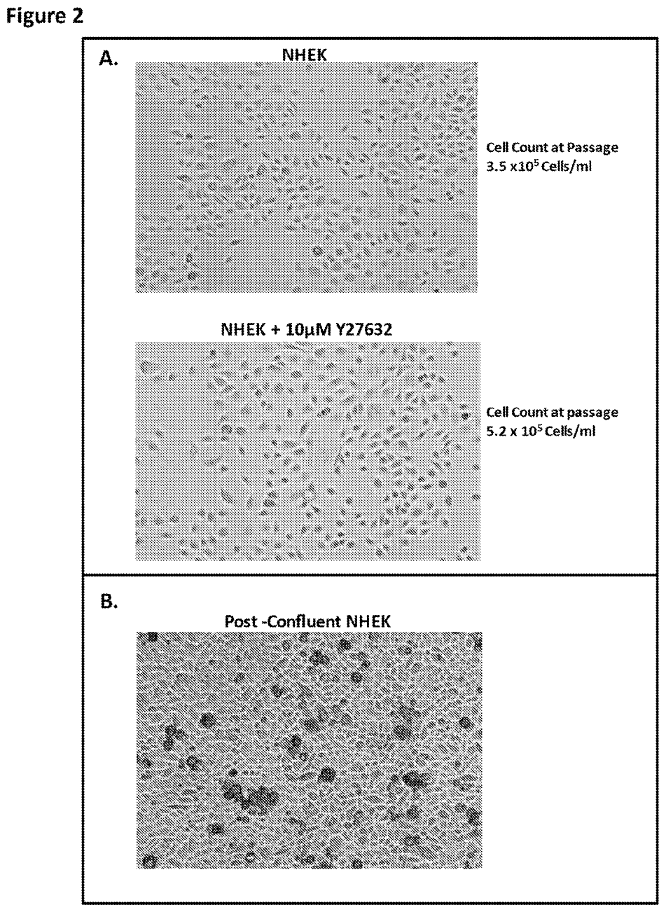

Neonatal human epidermal foreskin keratinocytes (NHEKs) were used to test the ability of compounds to reverse, arrest or slow down cellular ageing. These cells typically undergo approximately 35 cell divisions before the Hayflick limit is reached but they can be induced to undergo premature senescence and, what has been understood to be, irreversible growth arrest earlier than this by induction of cell-cell contact growth inhibition when cultured at high cell density/to confluence (Poumay & Pittelkow 1995 J Inv Dermatol 104, p 271-276). The inventors were thus inspired to culture post-confluent cells and assess the effect of test compounds on the growth, telomere loss and secretion of inflammatory factors by such cells.
An experimental schematic is provided as FIG. 1.
1.1 Culture of Early Passage NHEK's in the Presence and Absence of y27632
Pooled donor neonatal NHEK's (Cat No C-12005, Lot No 456Z001.1, PromoCell GmBH), serum-free PromoCell Keratinocyte Growth Medium 2 (KSFM, Cat No C20011. Lot No 472M197), CaCl₂) (Lot No 471055) and Supplements (Lot No 472M013) were all obtained from PromoCell GmBH. NHEK's were thawed directly into a single T75 in 20 mls of complete KSFM (CaCl2 & Supplements), incubated over night at 37° C., 5% CO₂ and the medium changed the following day.
After 3 days the cells were 80% confluent and were passaged (Ist) using 0.05% Trypsin+EDTA (Gibco) into ×2 T75 flasks at a split ratio of 1:4. After overnight incubation at 37° C., 5% CO₂, the cultures were at 30% confluence and 10 μM Y27632 (Cat No 688000, Lot No 2884132, Merck) was added to one of the flasks. After a further 48 hours both flasks were 60% confluent and phase contrast images were recorded (Zeiss Primavert, 4×/0.10 Mag) (FIG. 2A).
After a further 24 hours these cultures were subsequently passaged (2^{nd}) when at 80% confluence producing total live cell counts of 3.5×10⁵ cells/ml for KSFM and 5.2×10⁵ cells/ml for KSFM+10 μM Y27632. Fresh T75 flasks were seeded with 5.0×10⁵ cells from each in their respective medium. After 72 hours, these were close to confluent with cell counts of 3.7×10⁵ cells/ml for KSFM and 5.3×10⁵ cells/ml for KSFM+10 μM Y27632 at passage (3^{rd}) determined with Via-1 Cassette Viability and Cell Count device (NC3000 Image Cell Cytometer, Chemometec). Fresh T75 flasks were seeded, as described previously, and also 1.2×10⁴ NHEK cells/well were seeded into 24 well plates, incubated overnight to allow cell attachment, and the following subsequently added to separate wells:
A. NHEK in complete KSFM
B. NHEK+Y27632 in complete KSFM
C. Y27632 treated NHEK transferred to complete KSFM (Y27632 Removed)
D. NHEK transferred to complete KSFM+500 nM Tadalafil
E. NHEK transferred to complete KSFM+750 nM Tadalafil
F. NHEK transferred to complete KSFM+1000 nM Tadalafil
G. NHEK transferred to complete KSFM+1250 nM Tadalafil
H. NHEK transferred to complete KSFM+50 nM Fenbendazole
I. NHEK transferred to complete KSFM+100 nM Fenbendazole
J. NHEK transferred to complete KSFM+150 nM Fenbendazole
K. NHEK transferred to complete KSFM+200 nM Fenbendazole
   All drugs were added 24 hours after seeding. These cultures were then incubated at 37° C., 5% CO₂ under the above conditions for 6 days, with one medium change, until they were all confluent. The inventors assessed that these cells had stopped growing due to cell-cell contact inhibition and had become post-confluent as defined herein. FIG. 2B is an example of the appearance of post-confluent cells. All cultures, whether controls, Y27632 treated, tadalafil treated or fenbendazole treated appeared the same.

### 1.2 Effects of Y27632, Tadalafil and Fenbendazole on the Growth of Post-Confluent NHEK's:

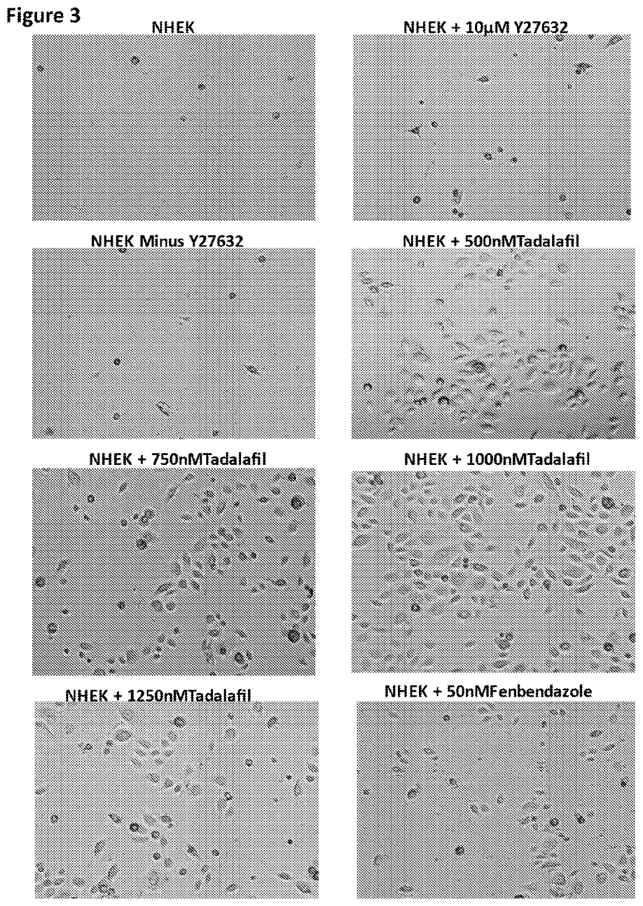

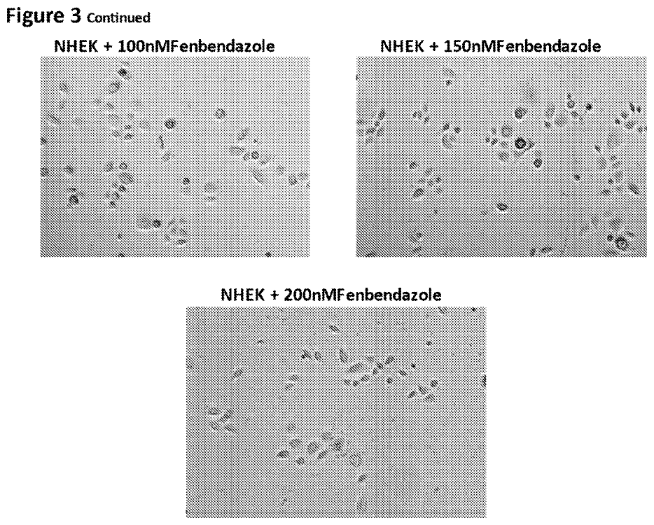

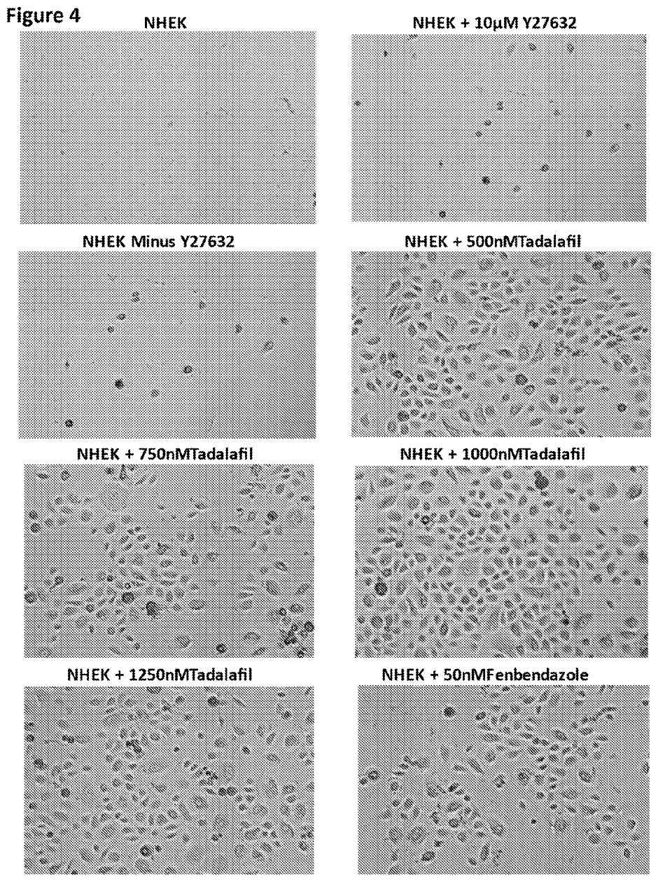

Post-confluent NHEK's (FIG. 2B) were subsequently passaged (4th) into a fresh 24 well plate using a split ratio of 1 in 4 in 2 mls of fresh complete KSFM. These were left overnight to attach and the same compounds added (as shown in A-K of 1.1) followed by incubation for a further 24 hours and subsequently photographed.
FIG. 3 clearly show that cells grown in the absence of Y27632 or test compound (FIG. 3: NHEK and A above) showed, as expected, no evidence of any continued post-confluent growth. Interestingly cells incubated in the presence of Y27632 (FIG. 3: NHEK+10 μM Y27632 and B above) after Passage 3 and 4; and cells incubated in the presence of Y27632 (FIG. 3: NHEK Minus Y27632 and C above) after Passage 3 (but for which Y27632 was removed after Passage 4) showed no, or minimal cell rejuvenation.
To the inventors' surprise, and in contrast to Y27632 treatments, cells treated with fenbendazole or tadalafil (D-K above) all exhibited cell growth. Optimal cell growth appeared to be caused by 100-150 nM Fenbendazole and 750-1000 nM Tadalafil.
The same 24 well plates that were photographed for FIG. 3, were cultured for a further 24 hrs and the typical appearance of cells is shown in FIG. 4. The surprising effects illustrated by FIG. 3 were confirmed with optimal cell growth appeared to be caused by 100-150 nM Fenbendazole and about 1000 nM Tadalafil.
These data indicate that both Tadalafil and Fenbendazole are more effective than Y27632 at rescuing cell-cell contact growth arrested NHEK's which clearly supports their ability to rejuvenate epidermal stem cells. Since the inventors, and others, have postulated that epidermal cells can be used as a model for stem cells found in other tissue types, this indicates this effect may translate to more general effects in vivo whereby Tadalafil and/or Fenbendazole may have the ability to promote self-renewal of other diverse cell types.

### 1.3 Effects of Tadalafil and Fenbendazole on the Growth of Post-Passage Confluent NHEK's

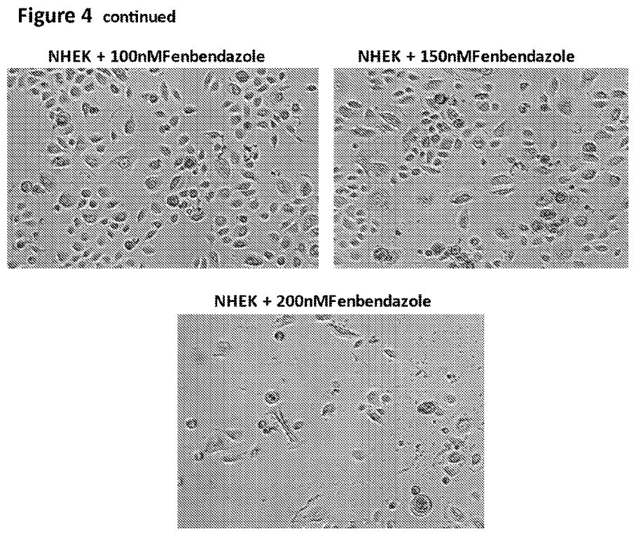

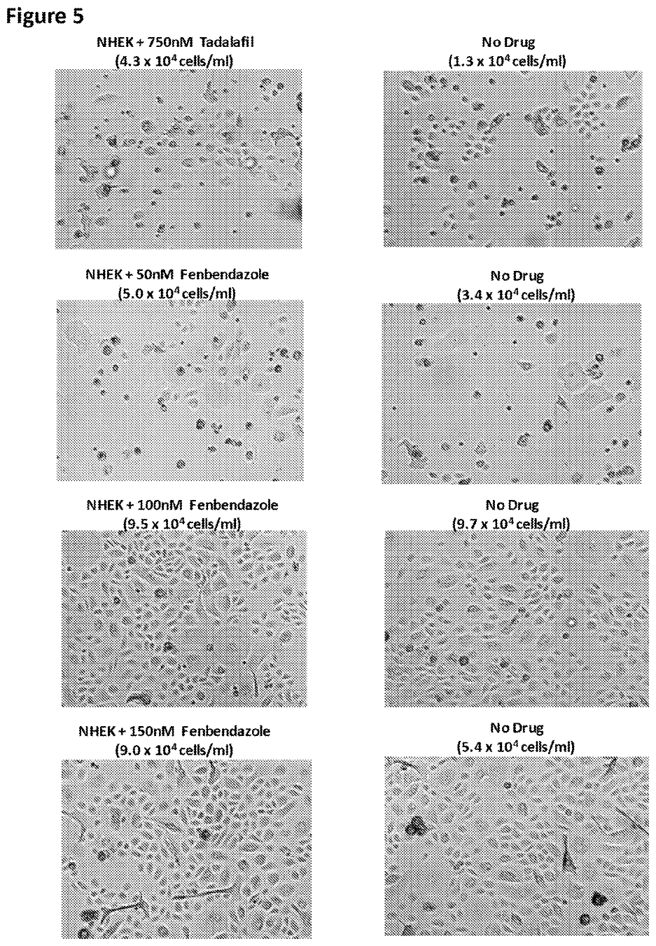

Surviving cells from the 24 well culture plate (FIG. 4), were passaged (5th) into separate T25 flasks, allowed to adhere overnight and then each respective drug treatment used in Example 1.1, was added for 5 days with one change of medium until all were post-confluent (i.e. as illustrated in FIG. 2B). These were then passaged (6th) and a tenth volume cells from each confluent flask seeded into two separate T25 flasks for each treatment A-K. All flasks were then incubated in complete KSFM overnight.
The first flask then received further treatments A-K and therefore received treatments for passages P3-P6 whereas the second flask received no further treatments. In other words the second flask only received treatments for P3-P5 with withdrawal of treatment following passage 6. These flasks were then incubated at 37° C. 5% CO₂ for 5 days with one change of medium. Photographs were then taken of each culture and cells counts were made.
It was found that for all concentrations of tadalafil that withdrawal of the drug resulted in a fall in cell number. This is illustrated in the top row of FIG. 5 where the number of viable cells fell from 4.3×10⁴ cells/ml to 1.3×10⁴ cells/ml (Via-1 Assay) following withdrawal of the PDE 5 inhibitor. This suggests that the beneficial effect of PDE 5 inhibitors may require continuous treatment.
Cell viability also dropped for some concentrations of Fenbendazole. However to the inventors' surprise the optimal concentration of fenbendazole from 1.3 was not associated with a loss in cell viability. This is illustrated in the third row of FIG. 5 where the number of viable cells (which were advantageously higher for this optimal concentration) remained about constant (9.5×10⁴ cells/ml and 9.7×10⁴ cells/ml) even if the benzimidazole compound with antihelminthic activity was withdrawn. This suggests that the beneficial effect of the benzimidazole compounds persist after drug withdrawal and advantageously means that continuous treatment with benzimidazole compounds is not required to reverse, arrest or slow down cellular ageing. These findings indicate that Fenbendazole was more effective than even Tadalafil at rescuing NHEK cells from cell-cell-contact-induced growth arrest. However, most significantly, the effects of Fenbendazole were seen to persist after drug withdrawal which is consistent with in vivo observations as illustrated by Example 2B where treatment of an 8 year old canine subject was discontinued for more than 2 years.

### 1.4 Effects of Y27632, Tadalafil and Fenbendazole on Late-Passage NHEK's:

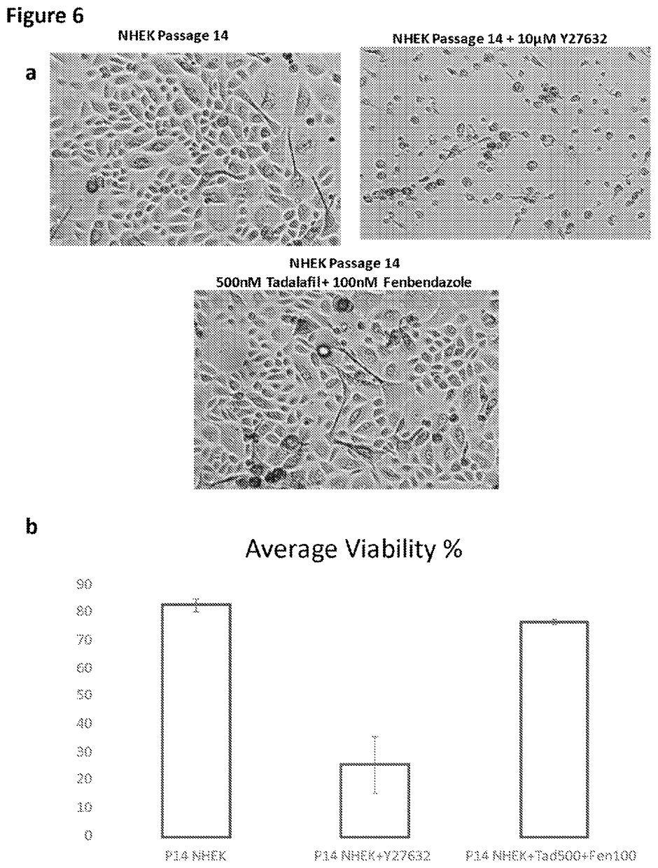

NHEK's were maintained in complete KSFM for 55 days with serial passage routinely carried out at 80% confluence in T75 flasks, as previously described in 1.1.
These cells were not grown to be post-confluent and the cells were therefore not used to assess the ability of test compounds to reverse, arrest or slow down cellular ageing. Instead, the cells were used to assess whether or not Y27632, fenbendazole or tadalafil has any toxic effects.
After the 55 days, a total of 50,000 cells/well of 14th passage NHEK cells were seeded into 6 well plates and, after 24 hours, Y27632, and a mixture of Tadalafil and Fenbendazole were added.
FIG. 6a shows images after incubation for 6 days in either complete KSFM (NHEK Passage 14); complete KSFM+10 μM Y27632 (NHEK Passage 14 +10 μM Y27632); or complete KSFM+500 nM Tadalafil+100 nM Fenbendazole (NHEK Passage 14 ++500 nM Tadalafil+100 nM Fenbendazole) with one medium change carried out during this period. Unlike early passage NHEK's treated with Y27632 late passage cells treated with Y27632 showed no proliferation and extensive cell death whereas the cells maintained in the presence of 500 nM Tadalafil and 100 nM Fenbendazole proliferated with little cell death. These results reinforce the observation that Fenbendazole (+/− Tadalafil) is more effective than Y27632 at extending the lifespan of NHEK cells irrespective of passage number.

### 1.5 Effects of Tadalafil and Fenbendazole on the Growth of Early-Passage Sub-Confluent NHEK's:

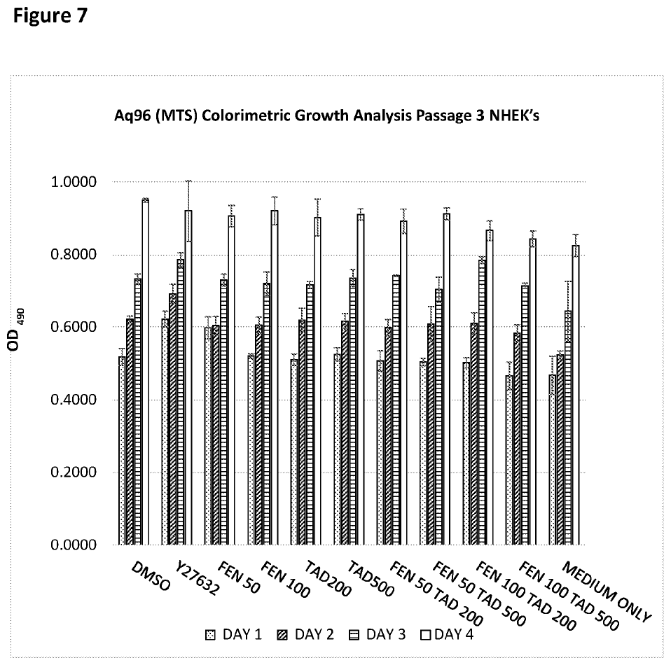

Sub-confluent passage 2 NHEK T75 flasks were cultured in complete KSFM maintained at 5% CO₂, 37° C. until ˜80% confluent. These were subsequently passaged (3^{rd}), accurate cell counts determined by Via-1 Cassette analysis and 8000 cells/well seeded in 200 μl of complete KSFM into 96 well microplates. Cells were allowed to adhere for 2 hrs at 5% CO₂, 37° C. followed by addition of DMSO only control, 10 μM Y27632 and a range of concentrations and combinations of Tadafil and Fenbendazole using three wells per data point. These were incubated for a total of 4 days with 20 μl of Aq96 (MTS) reagent (Promega UK) added to each replicate set of drug treatments every consecutive day followed by incubation for 1 hour at 5% CO₂. 37° C. OD readings were then taken at 490 nm using a Dynex Technologies MRX Revelation plate reader.
FIG. 7 shows the results of the MTS-based colorimetric proliferation assay carried on sub-confluent, passage 3, actively growing NHEK cells treated with: DMSO control: 10 μM Y27632; 50 nM and 100 nM Fenbendazole: 200 nM and 500 nM Tadalafil: 50 nM Fenbendazole+200 nM Tadalafil. 50 nM Fenbendazole+500 nM Tadalafil: 100 nM Fenbendazole+200 nM Tadalafil: 100 nM Fenbendazole+500 nM Tadalafil. These data demonstrate that none of the drug treatments caused any overt toxicity and, when compared to controls, produced marginal differences on the overall growth rate of actively growing NHEK cells.

### 1.6 Analysis of Average Telomere-Loss Per Population Doubling of Drug-Treated NHEK's

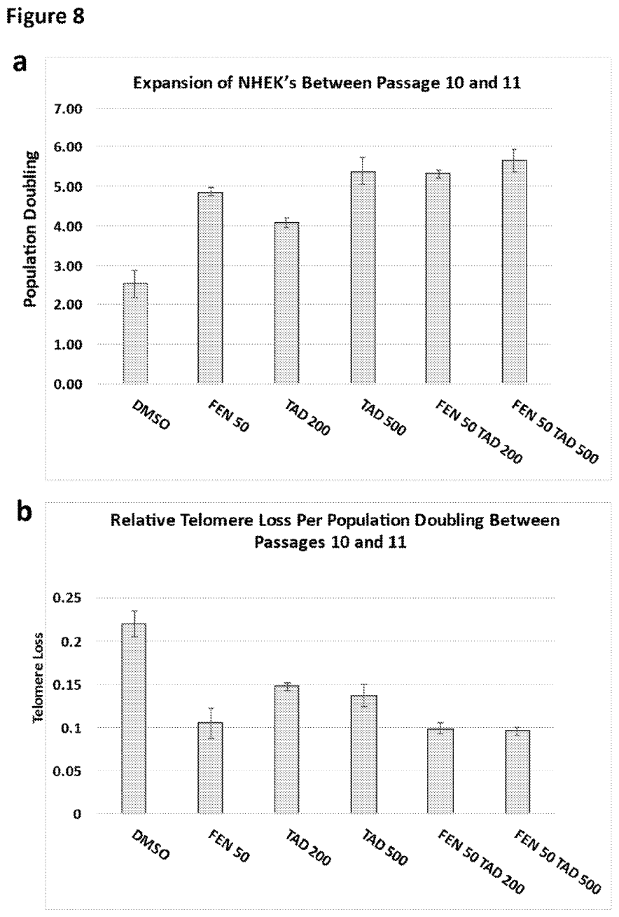

Sub-confluent passage 2 NHEK's in T75 flasks were cultured in complete KSFM maintained at 5% CO2, 37° C. until ˜80% confluent as described in 1.5. These were subsequently passaged and accurate cell counts determined by Via-1 cassette NC3000 Image Cell Cytometer. An aliquot of 40,000 cells was then seeded into T25 flasks which were incubated for 24 hours in KKSFM at 5% CO₂, 37° C. to allow cells to adhere. Fenbendazole and Tadalafil were added to each of these as single agents, or in combination, and the cells allowed to expand for 4 days at 5% CO₂, 37° C. when they had reached approx 20% confluency after which both medium and drugs were replaced. These cultures were then incubated for another 3 days at 5% CO2, 37° C. when confluency was approx 80%. At this point, cells were harvested, Via-1 counts carried out, 50,000 cells pelleted for DNA preparation (DNeasy® Blood & Tissue Kit, Qiagen UK, Cat No 69505) and 40,000 cells seeded into a fresh T-25 flask for each specific drug treatment. The whole procedure was then repeated 8 times until passage 11. Extracted DNA quality and quantity was determined with a QuickDrop DNA Spectrophotometer (Molecular Devices. Model 504 7178) and then this was used to analyse comparative telomere length in each sample by use of a Relative Human Telomerase Length Quantification qPCR Assay Kit (ScienCell™, Cat No 8908) with a Stratagen qPCR/Real Time PCR Machine. Model Mx3005P®.
Via-1 cell counts between passages 3 and 10, demonstrated no difference in cell population doubling rates between any of the drug treatments or controls which is consistent with the short-term growth analysis shown in FIG. 7. Analysis of relative telomere length was carried out on DNA extracted from passages 3-10 in triplicate using a qPCR Assay Kit (ScienCell™, Cat No 8908), according to the manufacturer's instructions. This showed no consistent difference in telomere length between any of the drug treatments (Data not shown).
However, analysis of Via-1 cell counts between passage 10 and 11 showed a significant reduction in proliferation of the DMSO control when compared to drug treated cells (FIG. 8a) where the results were calculated as the number of cell population doublings per treatment group. Comparative telomere length analysis on DNA extracted from passage 10 and 11 NHEK's, showed that the relative telomere loss per population doubling was significantly reduced in the drug treated cells with, either Fenbendazole as a single agent or when combined with Tadalafil, producing the greatest reduction (FIG. 8b).
These observations indicate that the various drug treatments evaluated all have the potential to extend the lifespan of cultured primary NHEK's

### 1.6 Analysis of the Effects of Fenbendazole and Tadalafil on the Production of Interleukin 6 (IL-6) by NHEK's

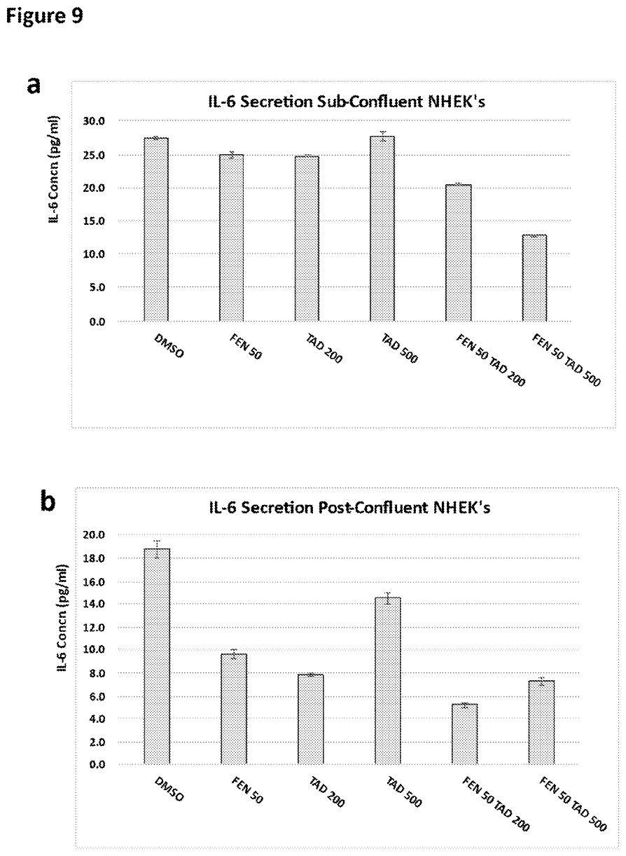

IL-6 is a pro-inflammatory cytokine which is also known to be a key member of the senescence associated secretory phenotype (SASP) group of proteins which are know to promore “inflammaging” (Khavinson et al. (2022) Cells 12 (1): 106 doi: 10.3390/cells12010106).
Aliquots of 60,000 NHEK cells were seeded into separate T25 flasks in complete KSFM and incubated at 5% CO₂, 37° C. for 3 days until they reached 20% confluence. Fenbendazole and Tadalafil were then added to each of these as either single agents, or in combination, and the cells incubated for a further 3 days at 5% CO₂, 37° C. until the cells reached >50% confluence. A sample of medium was then taken for analysis of IL-6 levels using a Human IL-6 SimpleStep ELISA Kit (AbCam Cat No ab178013) according to the manufacturer's instructions (FIG. 9a). Both medium and drugs were replaced, the cells incubated for a further 3 days until confluent, medium and drugs were changed and the incubation continued for 4 days post-confluence when final medium samples were taken for analysis of IL6 levels (FIG. 9b). Each data point was repeated in triplicate and OD 490 nm readings were determined with a Dynex MRX Plate reader.
Compared to DMSO control, a modest reduction in the secretion of IL-6 was observed for sub-confluent NHEK's treated with combinations of Tadalafil and Fenbendazole (FIG. 9a). This effect was enhanced in post-confluent NHEKs where all drug-treated cells showed reduced secretion of IL-6. The least effective was 500 nM Tadalafil and the most effective, with an approximate 4 fold reduction in the secretion of IL6, was 50 nM Fenbendazole and 200 nM Tadalafil combined (FIG. 9b).
These results are consistent with all the drug treatments tested inducing a drop in IL-6 secretion from post confluent cultures of NHEks which supports their ability to reduce inflammation and this “inflammaging”.
In summary, the data present in Example 1 demonstrates the use of Fenbendazole both as a single agent, and when combined with Tadalafil, as a means of prolonging the lifespan of cultured primary human NHEK's by preventing; irreversible cell-cell-contact induced growth-arrest: cell senescence and inhibiting secretion of pro-inflammatory IL-6. Furthermore, the effects of Fenbendazole were seen to persist after the drug was withdrawn.

### Example 2

Long-term canine studies were conducted on two dogs (C2A and C2B) to test the efficacy of compositions that may be used according to the invention.

### 2.1 Canine Case Study Subject C2a:

In 2017, a 10 yr old (D.O.B Jan. 5, 2008) female Lhasa Apso/Jack Russell/Shih Tzu cross-breed (confirmed by genetic breed analysis: DNA-My-Dog) with a body mass of 9 Kg, was treated with 0.6 gm/day of 22.2% Fenbendazole (Panacur® granules) which was given orally for 3 consecutive days/week for 1 month and this cycle was repeated every 3^{rd} month giving 4 cycles/annum providing a total of 16×1 month treatment cycles spread over a period of 4 yrs up to the present day.

### 2.1.1 Impact on Epigenetic or Biological Age of Subject C2A

Subject C2A's chronological age was verified by microchip implantation date using the Petlog database and, in March 2022, a cheek epithelium swab was sent to DNA-My-Dog for genetic assessment of breed composition and biological age by analysis of breed-specific telomere length. The results, returned on the 30 Mar. 2022, reported a biological age of 3 yrs old. Three more treatment cycles were subsequently administered and a repeat biological age telomere test carried out on the 30 Sep. 2022 which also returned a biological age of 3 yrs old. These data indicate a persistent reduction in biological age of 11 yrs which equates to approximately an 80% reduction in biological age over 4 years. Translating these findings to humans, a 14 yr old Lhasa Apso has an equivalent human age of about 72 whereas a 3 yr old has an equivalent human age of about 28. This indicates a potential reduction in human age of 44 yrs which would equate to a 61% reduction of biological age in humans.

### 2.1.2 Efficacy Against Age Related Pathologies in Subject C2A

At the start of the 4 yr course of treatment it was noted that the canine subject had numerous benign skin conditions, was overweight and largely inactive. As the aforementioned treatment progressed, significant weight-loss and increased physical activity was noted in addition to normalization of skin and regression of subcutaneous lipoma like lesions with no evidence of age-associated hair-greying observed. Furthermore, no evidence of teeth dis-colouration was noted and there was also evidence of the activation of new growth of teeth in addition to accelerated growth of nails and hair.

### 2.2 Canine Case Study Subject C2B

In 2017, a 4 yr old Jack Russell/Poodle/Maltese cross-breed (Confirmed by genetic breed analysis: DNA-My-Dog) with a body mass of 9.2 Kg was treated with 1.0 gm/day of 22.2% Fenbendazole Panacur® granules for 3 days per week and 4 days without. The dosing schedule of 3 days with, 4 days without treatment per week was continued for 3 months after which the dose was reduced to 0.6 gm/day of Panacur® and treatment alternated between 1 month with and 1 month without for 19 months providing a total of 11×1 month treatment cycles over the whole time period. After this, treatment was discontinued for 2 years and 5 months up to 12 May 2022 when a cheek swab was taken for analysis of biological age and this was repeated 5 months later on the 30 Sep. 2022.

### 2.2.1 Impact on Epigenetic or Biological Age of Subject C2B

The initial reason for treating this animal was that it had been diagnosed with an anal sac adenocarcinoma and, at the time, it was decided to explore the use of Fenbendazole as an alternative treatment. Most notably, the tumour regressed during the treatment and has not recurred since. Subject C2B's birth date was May 2014 giving a chronological age of 8 yrs which was verified by microchip implantation date using the Petlog database. Following the outlined treatment with Fenbendazole, in April 2022, a cheek epithelium swab was sent to DNA-My-Dog for assessment of breed composition and biological age by analysis of breed-specific telomere length. The result, returned on the 12 May 2022, reported a biological age of 1 yr old indicating a reduction in biological age of 7 yrs. which is equivalent to ˜90% reduction in biological age. Since the total treatment time was 19 months followed by an interval of 2 yrs 5 months prior to the first telomere age test on the 12 May 2022, this indicates that the anti-ageing effects persist for some time after an episode of treatment. After a further gap of 5 months without treatment, on the 30 Sep. 2022, a repeat telomere length analysis on subject C2B now showed an increase of 2 yrs to a biological age of 3 yrs old. However, unlike subject C2A, no treatment was given during this period so the most likely explanation is that the efficacy time-limit of the initial treatment has been exceeded.

### 2.2.2 Efficacy Against Age-Related Pathologies in Subject C2B

Similar to subject C2A, subject C2B does not show any visible signs of ageing such as greying hair, discolouration of teeth, signs of cataract formation and is extremely healthy and active. C2B also shows new growth of teeth, nails and hair. Most surprisingly, subject C2B indicates that the anti-ageing effects of Fenbendazole persist for at least 2 years after the treatment is discontinued which is consistent with the observation that its growth promoting effects on NHEK cells in vitro were seen to persist when the drug was withdrawn.

### Example 3

Short-term human case studies were conducted on three subjects (H3A, H3B and H3C) to test the efficacy of compositions that may be used according to the invention.

### 3.1 Human Case Study Subject H3A

This subject is a 68 yr old (in 2022) male human volunteer. He is a non-smoker; 180 cm in height with a body mass of 70 Kgm (BMI 21.5); has low-alcohol consumption (<7 Units/week); leads a healthy lifestyle; eats a Mediterranean diet and carries out an identical vigorous exercise routine for 1 hr/day.
Subject H3A commenced taking 5 mg/day of Tadalafil on the 16 Jun. 2017 and has continued to do so since that time.
In April 2022, a saliva sample was sent to Elysium Health for epigenetic analysis of biological age by use of their Index test (www.elysiumhealth.com/products/index) date stamped 3 Mar. 2022.
In addition to Tadalafil, on the 9 May 2022, H3A commenced taking 1.2 gm/day of Panacur® granules for 5 days/week for 4 weeks, 4 days/week for 4 weeks and 3 days/week for 2 weeks giving a total duration of 10 weeks. A repeat saliva sample was taken on the 10 Mar. 2023 and sent to Elysium Health for Index epigenetic analysis of biological age.
In addition to the Elysium Index test, a comparative telomere length analysis (See Section 1.6) was also carried out on DNA extracted from passive saliva, obtained from subject H3A, on the 2 Aug. 2022 and 23 Mar. 2023 using a DNeasy® Blood & Tissue Kit (Qiagen UK, Cat No 69505). This material was checked for purity and quantified with a QuickDrop Spectrophotometer (Molecular Devices, Model 504 7178).
Furthermore, during treatment and post-treatment follow-up, records were made of its impact on any pre-existing age-related conditions.

### 3.1.1 Impact on Epigenetic Biological Age of Subject H3A

After a 4 yr period of taking Tadalafil and prior to commencing Panacur® treatment on the 9 May 2022 an Elysium Index test reported a biological age of 57 yrs for subject H3A which equates to an ageing rate of 0.84 as determined by dividing biological age with chronological age. Eight months later, after a 10 week treatment of Tadalafil combined with Fenbendazole (Panacur Granules), subject H3A had a biological age of 58 yrs although this produced no change in the subjects ageing rate of 0.84 since the subjects chronological age was now 69 yrs.

### 3.1.2 Efficacy Against Age-Related Pathologies in Subject H3A

Subject H3A noted improved mood and mental acuity and also noticed a marked improvement in cardiac response to exercise within a few days of commencing treatment with Panacur® (Fenbendazole). After completing the same 40 minute vigorous aerobic exercise regime, peak heart rate had decreased from 130-135 bpm to 118-124 bpm (Polar Chest Strap Monitor) and the exercise routine was noticeably easier. Furthermore, this improvement persisted for the entire period of treatment and for the 10 months follow up. Within 4 weeks of commencing treatment with Panacur®, a significant increase in growth of hair and nails was noted and a developing bald patch on the H3A's pate has now gone. Furthermore, for >10 yrs subject H3A had experienced a painful stiff neck with pronounced crepitus on rotation. After the combined Tadalafil and Fenbendazole treatment, neck pain has completely resolved with substantially reduced crepitus. Furthermore, H3A's visual acuity had also increased with significantly improved ability to read small-print.

### 3.2 Human Case Study Subject H3B

This subject is a 42 yr old (in 2022) male human volunteer. He is a former smoker: 180 cm in height with a body mass of 108 Kg (BMI 33.3); with moderate alcohol consumption (14 Units/week): leads a reasonably healthy lifestyle; eats an Asian style diet; and takes moderate exercise for 30 min/day. In April 2022, a saliva sample was sent to Elysium Health for epigenetic analysis of biological age by use of their Index test.
On the 9 May 2022 H3B commenced taking 5 mg/day of Tadalafil which was continued as a single agent for 5 weeks. On the 6 Jun. 2022 H3B commenced taking 1.2 gm/day of Panacur® granules for 3 days/week which was continued for 8 weeks. Following this, in March 2023, a repeat saliva sample was taken and sent to Elysium Health for Index epigenetic analysis of biological age.
Furthermore, in addition to the Index tests, comparative telomere length analysis was carried out on DNA extracted from passive saliva samples from subject H3B on the 2 Aug. 2022 and 23 Mar. 2023 using a DNeasy® Blood & Tissue Kit (Qiagen UK, Cat No 69505). This material was checked for purity and quantified with a QuickDrop Spectrophotometer (Molecular Devices, Model 504 7178). This

### 3.2.1 Impact on Epigenetic Biological Age of Subject H3B

Prior to commencing treatment in early May 2022, Elysium reported a biological age of 40 yrs for subject H3B which equates to a biological age 5% less than chronological age equivalent to a 0.97 rate-of-aging. Results of the second Index test, carried out approximately 1 year later in April 2023, showed H3B still had a biological age of 40 yrs but was now chronologically aged 43 yrs which is equivalent to a reduced rate-of-aging of 0.95.

### 3.2.2 Efficacy Against Age-Related Pathologies in Subject H3B

H3B noted improved mood and mental acuity within a few days of commencing combined Tadalafil/Fenbendazole treatment. Furthermore, subject H3B also had a large, historic, injury-related varicocele since aged 10 yrs old which had not shown any improvement since that time. Following treatment the varicocele has resolved and been superseded by a uniform expansion of the volume of the effected testicle. Additionally, two years previously H3B developed a large discoloured (0.8 cm diameter) mole-like growth on the side of the forehead. This has now completely resolved post-treatment.

### 3.3 Impact on Relative Telomere Length of Subjects H3A and H3B

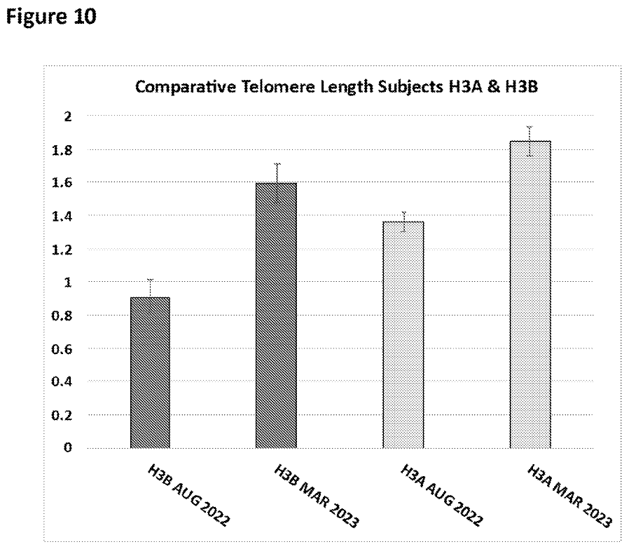

The results of comparative telomere length analysis carried out on DNA extracted from saliva from subject H3A and H3B immediately and 8 months after treatment with Tadalafil and Fenbendazole are shown in FIG. 10. The same quantitative telomere PCR method described in Section 1.6 was used with the exception that each assay was carried with 6 rather than 3 replicates. All results were calculated relative to the sample with the shortest telomere length (August 2022, Subject H3B) and clearly show a significant increase in relative telomere length occurring approximately 8 months after treatment with Tadalafil and Fenbendazole. Subject H3B showed the greatest increase with teleomere length nearly doubling in the post-treatment period.
Comparison of telomere length data to methylation-based Elysium Index results obtained for subjects H3A and H3B, show a more pronounced post-treatment difference with telomere analysis which may reflect differences between the two analytical procedures. Indeed these two methods are known to have a modest correlation with predicted phenotypic age (Pearce et al. (2022) GeroScience 44 (3) p 1861-1869).

### 3.4 Human Case Study Subject H3C

This subject is a 58 yr old (in 2022) male human volunteer. He is a non-smoker: 189 cm in height with a body mass of 81 Kgm (BMI 22.5): low alcohol consumption (<7 Units/week): leads a healthy lifestyle: eats a Mediterranean style diet: is a former national athlete and carries out vigorous exercise for >1 hr/day. H3C has a family history of coronary arterial disease and has a highly-calcified focal lesion in the left anterior descending artery with 25-60% stenosis in the proximal portion. He also has early-stage benign prostatic hyperplasia (BPH) and stage 4+ osteoarthritis (OA) in the right knee and stage 3 in the left. His right knee was judged as having bone-on-bone plus joint-degradation and was treated with a menisectomy in 1994 followed by an osteotomy in 2021 and finally a micro-fragmented adipose tissue (MFAT) injection in 2021.
In May 2022, a saliva sample was sent to Elysium Health for epigenetic analysis of biological age by use of their Index test and on the 31 May 2022 H3C commenced taking 5 mg/day of Tadalafil which was continued as a single agent for >8 weeks. In August 2022, this was combined with 222 mg/day of Fenbendazole (Fenben Lab, Canchema Ltd, Lithuania) which, as of June 2023, is still ongoing. No other changes in lifestyle or medication were carried out during this period.

### 3.4.1 Impact on Epigenetic or Biological Age of Subject H3C

Prior to commencing treatment in early on the 28 May 2022, Elysium reported a biological age of 44 yrs for subject H3C which equates to a biological age 24% less than chronological age equivalent to a rate-of-aging of 0.77. Results of the second Index test, carried out approximately 8 months later on the 4 Apr. 2023, showed H3C still had a biological age of 44 yrs but was now chronologically aged 59 yrs which equates to a biological age 25% less than chronological age equivalent to a reduced rate-of-aging of 0.76.

### 3.4.2 Efficacy Against Age-Related Pathologies in Subject H3C

H3C had a coronary artery calcification (CAC) index of 425 in December 2021 (CT scan) which had reduced to 303 by February 2023 and equates to a post-treatment improvement of ˜30%. This was coupled with zero progression in plaque number and size and was associated with a reduction in plaque burden in the left anterior descending artery. Furthermore, in March 23, H3C's blood biochemistry had undetectable levels of CRP and his blood glucose had reduced from 108 to 97 mg/dl. His HDL cholesterol has increased from 42 to 47 mg/dl and his triglycerides have reduced from 82 to 61 mg/dl post-treatment.
H3C's right knee OA has completely resolved without any pain or inflammation in spite of an extensive exercise regime of running, hill-training, cycling and rowing. The improvement has been confirmed by a positive MRI scan with no detectable degenerative changes. Indeed, these observations are consistent with the combined Fenbendazole/Tadalafil treatment acting to facilitate the growth and survival of MFAT derived stem cells which subsequently enhance repair of the joint capsule, and/or help to protect the MFAT graft patency and its anti-inflammatory and cell protective properties.
H3C has trained hard since aged 12 and his fitness level at maximal exertion has improved by approximately 10% in the last 12 months including VO2 max and maxed out training times on monitored routes (Garmin Fitness Tracker). Other findings include weight loss from 84 Kg to 80 Kg over the period with no muscle loss and possibly a higher percentage of darker hair in the beard region and scalp.

### Example 4

Short terms studies were conducted on rodents to test the efficacy of compositions that may be used according to the invention.

### 4.1 Methods

Seventy-eight Sprague-Dawley rats aged either 16 or 22 months were maintained with daily 12-hour light/dark cycles and allowed constant access to feed and water. The animals were bred and housed at the Cantacuzino National Medico-Military Institute for Research and Development, Bucharest 050096, Romania. These animals were split into 3 separate groups of 26, each containing the same number of younger and older animals, which were then given the following feeds prepared by Altromin Spezialfutter GmbH & Co. for a period of 4 months:
(A) Control maintenance feed plus standard multivitamin supplement (No API's)
(B) Formulation (A) plus 80 mg/Kilo of Fenbendazole.
(C) Formulation (B) plus 12.5 mg/Kilo Tadalafil.
   A buccal swab was then taken from each animal using an OracollectDNA OCR-100 sampling device (DNA Genotek, Canada). DNA was extracted using a DNeasy® Blood & Tissue Kit (Qiagen UK, Cat No 69505) which was checked for purity and quantified with a QuickDrop Spectrophotometer (Molecular Devices, Model 504 7178). This material was then used to analyse relative telomere length as described in section 1.6 except that the Relative Rat Telomere Length Quantification qPCR Assay Kit (ScienCell Research Labs, Catalog No. R8908) was used. Five consecutive samples from each of the three treatment groups were analysed on each 96 well PCR plate to minimize any inter-plate variability.

### 4.2 Results

### 4.2.1 Mortality

After 4 months of treatment, there were 18, 23 and 22 surviving animals in groups (A), (B) and (C) which equates to 30.7%, 11.5% and 15.4% mortality in each group respectively. This observation is consistent with an increase in the life-expectancy of animals treated with medicated feeds (B) or (C) when compared to those in control feed group (A).
The animals were given free access to the feed and animals typically ate between 20 and 40 g of feed/day. This equated to a a dose of approximately 1.5-3.0 mg/animal/day of Fenbendazole and a dose of approximately 0.25-0.5 mg/animal/day of Tadalafil.
It was noted that the appetite was greater in the treated animals (groups (B) and (C)).

### 4.2.2 Impact on Relative Telomere Length Between Treatment Groups

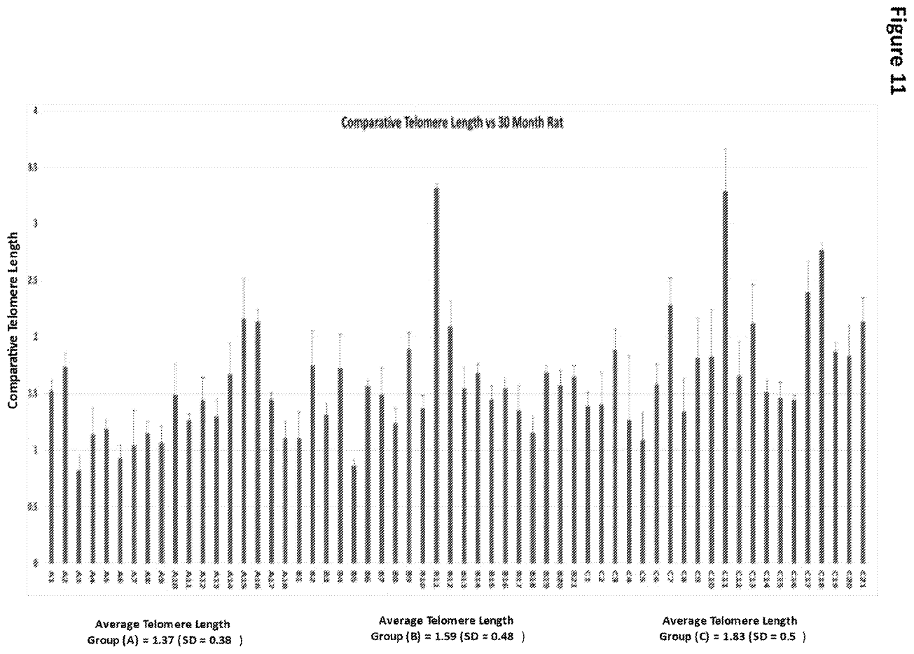

The results of comparative telomere length analysis carried out on DNA extracted from 60 buccal swabs taken from rats following 4 months administration of either feeds (A), (B) or (C) are shown in FIG. 11. Although inter-animal variability was observed within each group, it is notable that the average telomere length for control group (A) was the shortest with an average of 1.37 (SD=0.38) times that found in DNA extracted from a 30 month old comparator. The average age of the animals in group (A) was younger than the 30 month old comparator and it was therefore expected that the telomere length of this group would be longer than for the comparator. Using the same analytical approach, group (B) animals treated with Fenbendazole as a single agent had the next longest at 1.59 (SD=0.48) whereas group (C) animals treated with Fenbendazole+Tadalafil had the longest telomeres at 1.83 (SD=0.5) which equates to a 37% increase above that of the control group (A) and indicates that the two drugs combined had the greatest efficacy.
These data illustrate that rats treated with APIs according to the invention had reduced mortality, better appetites and longer telomeres than the control group (A). This demonstrates that compositions comprising a benzimidazole compound with antihelminthic activity (optionally combined with, or co-administered with, a Phosphodiesterase 5 inhibitor) may be used for reversing, arresting or slowing down cellular ageing in a vertebrate subject.

1. A composition comprising a benzimidazole compound with antihelminthic activity for use as a medicament to reverse, arrest or slow down cellular ageing in a vertebrate subject.

2. The composition for use according to claim 1 wherein the benzimidazole compound with antihelminthic activity is selected from the group comprising: Fenbendazole, Mebendazole, Flubendazole, Parbendazole, Oxfendazole, Oxibendazole, Albendazole, Ricobendazole, Albendazole sulfoxide, Thiabendazole, Thiophanate, Febantel, Netobimin, Triclabendazole and functional equivalents thereof.

3. The composition for use according to claim 2 wherein the benzimidazole compound with antihelminthic activity is Fenbendazole.

4. The composition for use according to any preceding claim further comprising a Phosphodiesterase 5 inhibitor.

5. The composition for use according to any one of claims 1-3 wherein the composition is used as a medicament to reverse, arrest or slow down cellular ageing in a vertebrate subject and wherein the composition is co-administered with another composition comprising a Phosphodiesterase 5 inhibitor.

6. The composition or compositions for use according to claim 4 or 5 wherein the Phosphodiesterase 5 inhibitor is selected from the group comprising Vardenafil, Sildenafil, Avanafil, Tadalafil and functional equivalents thereof.

7. The composition or compositions for use according to claim 6 wherein the Phosphodiesterase 5 inhibitor is Tadalafil.

8. The composition or compositions for use according to any one of claims 1-7 wherein reversing, arresting or slowing down cellular ageing results in a reduction in the epigenetic or biological age of the subject.

9. The composition or compositions for use according to claim 8 wherein the epigenetic or biological age of the subject is determined by measuring telomere length.

10. The composition or compositions for use according to any one of claims 1-9 wherein reversing, arresting or slowing down cellular ageing treats or prevents an age-related disease.

11. The composition or compositions for use according to claim 10 wherein the age-related disease is selected from the group comprising: Type II diabetes, skin wrinkles, varicosities, hair-loss, tooth loss/wear, joint-degeneration, cardiovascular disease, muscle loss, macular degeneration, neurodegenerative conditions and decreased immune function.

12. The composition or compositions for use according to any one of claims 1-9 wherein reversing, arresting or slowing down cellular ageing promotes regeneration of damaged tissue.

13. The composition or compositions for use according to claim 12 wherein regeneration of damaged tissue is promoted following spinal injury or a burn.

14. The composition or compositions for use according to any one of claims 1-9 wherein the subject has received or is receiving a cancer therapy.

15. The composition or compositions for use according to any preceding claim used in a dosing regimen wherein the benzimidazole compound with antihelminthic activity is administered as an oral dose of between 1 and 70 mg/Kg/day.

16. The composition or compositions for use according to any one of claims 4-14 in a dosing regimen wherein the Phosphodiesterase 5 inhibitor is administered as an oral dose of between 10 and 150 ng/kg/day.

17. The composition or compositions for use according to claim 15 or 16 wherein the composition or compositions are administered as continuous or discontinuous treatment regimens of 1-7 days/week for intermittent periods of between 1-12 months in any 12 month period.

18. The composition or compositions for use according to any preceding claim wherein the subject is an animal of veterinary interest.

19. The composition or compositions for use according to any one of claim 1-17 wherein the subject is a human being.

20. A method of reversing, arresting or slowing down cellular ageing in a vertebrate subject in need of such treatment comprising administering a therapeutically effective amount of a composition or compositions as defined in any one of claims 1-19.

21. A composition formulated for pharmaceutical, veterinary or nutraceutical use comprising a benzimidazole compound with antihelminthic activity and a PDE 5 inhibitor.

22. The composition according to claim 21 comprising;
(i) a Benzimidazole compound with antihelminthic activity selected from the group comprising: Fenbendazole, Mebendazole, Flubendazole, Parbendazole, Oxfendazole, Oxibendazole, Albendazole, Ricobendazole, Albendazole sulfoxide, Thiabendazole, Thiophanate, Febantel, Netobimin, Triclabendazole and functional equivalents thereof; and
(ii) a Phosphodiesterase 5 inhibitor selected from the group comprising Vardenafil, Sildenafil, Avanafil, Tadalafil and functional equivalents thereof.

23. The composition according to claim 22 comprising Fenbendazole and Tadalafil.
